(19) United States
(12) Patent Application Publication
(10) Pub. No.: US 2018/0185286 A1
(43) Pub. Date: Jul. 5, 2018

(54) POROUS MATERIALS CONTAINING COMPOUNDS INCLUDING PHARMACEUTICALLY ACTIVE SPECIES

(71) Applicants: NOVARTIS PHARMA AG, Basel (CH); MASSACHUSETTS INSTITUTE OF TECHNOLOGY, Cambridge, MA (US)

(72) Inventors: Allan Stuart Myerson, Boston, MA (US); Xiaochuan Yang, Greenbelt, MD (US); Marcus O'Mahony, Cambridge, MA (US); Markus Krumme, Allschwil (CH); Norbert Rasenack, Loerrach (DE); Leia Dwyer, Providence, RI (US)

(73) Assignees: NOVARTIS PHARMA AG, Basel (CH); MASSACHUSETTS INSTITUTE OF TECHNOLOGY, Cambridge, MA (US)

(21) Appl. No.: 15/301,221

(22) PCT Filed: Mar. 31, 2015
(86) PCT No.: PCT/US2015/023592

§ 371 Date: Sep. 30, 2016

Related U.S. Application Data
(60) Provisional application No. 61/972,780, filed on Mar. 31, 2014.

(51) Int. Cl. A61K 9/14; A61K 9/20; A61K 9/48; A61K 31/192; A61K 31/216; A61K 31/343; A61K 9/16

(52) U.S. Cl. CPC A61K 9/143; A61K 9/2009; A61K 9/485; A61K 9/1652; A61K 31/216; A61K 31/343; A61K 31/192

(57) ABSTRACT

Materials containing pharmaceutically active species in solid (e.g., crystal) form, and related methods, are provided, allowing for improved stability, solubility, bioavailability, and/or dissolution rates for pharmaceutically active species having poor aqueous solubility.

### RELATED APPLICATIONS

This application claims the benefit of U.S. Provisional Patent Application Ser. No. 61/972,780, filed Mar. 31, 2014, entitled “Porous Materials Containing Compounds Including Pharmaceutically Active Species,” which is incorporated herein by reference in its entirety.

### FIELD OF THE INVENTION

Embodiments related to porous materials including pharmaceutically active species are provided.

### BACKGROUND OF THE INVENTION

In order to improve the solubility/dissolution rate or bioavailability of pharmaceutically active species (or active pharmaceutical ingredients or APIs) which exhibit poor aqueous solubility, formulation of such compounds often utilize co-solvents or high surfactant concentrations, both of which can have adverse side effects. For example, co-solvents such as propylene glycol can result in system toxicity, while hypersensitivity reactions have been observed for formulations (e.g., taxol formulation) using the surfactant cremophore EL®. The formulation of lipophilic drugs using mixed micelles to produce microemulsions often requires the use of high concentrations of surfactant. Inclusion complexes, such as cyclodextrins, have also been used to formulate drugs with poor aqueous solubility, as in the case of itraconazole, but this technology is limited insomuch as the API must fit the molecular cavity offered by the cyclodextrin and the final formulation contains a high level of excipient. In summary, these current formulations are complex multicomponent systems that can have adverse in vivo reactions.

Technologies also exist which aim to generate nanosized particles as a means of improving solubility/dissolution rate or bioavailability of APIs via a rapid anti-solvent precipitation process, by high pressure homogenization (HPH), or by rapid expansions of supercritical fluids containing API molecules. These technologies can involve complex procedures like lyophilization to maintain particle size in a suspension or in the case of HPH that the API powder be suspended in a solution containing high levels of surfactant. Supercritical fluid technologies have been used to generate API nanoparticles but their production is also highly dependent on the use of water-soluble polymeric stabilizers in addition to processing with cosolvents. Other processes involve a milling step (e.g., jet milling) to produce API nanoparticles. However, milling often results in the loss of crystallinity, conversion to amorphous material, and/or contamination. In each of these technologies at least one more additional processing step is typically required to formulate such products.

Other technologies related to nanosized API particles are described in U.S. Publication No. 2006/127480, which describes pharmaceutical excipients comprising inorganic particles in association with an organic polymeric material, and U.S. Publication No. 2009/0130212, which describes the preparation of small particles containing pharmaceutical drugs.

### SUMMARY OF THE INVENTION

Various methods, compositions, and formulations are provided.

Some embodiments provide methods for forming a material comprising a pharmaceutically active species. In some embodiments, the method may comprise contacting a porous material comprising a plurality of pores with a pharmaceutically active species, such that the pharmaceutically active species enters the pores; placing the porous material under a set of conditions which facilitates formation of a crystal of the pharmaceutically active species; and allowing the pharmaceutically active species to form a crystal within the plurality of pores, wherein, upon formation of the crystals within the plurality of the pores, the exterior surface of the porous material is substantially free of crystals of the pharmaceutically active species having a size of 1 micron or greater.

In some embodiments, the method further comprises filtering and/or washing the porous material before formation of the crystal. In some embodiments, the method further comprises filtering and/or washing the porous material after formation of the crystal. In some cases, the exterior surface of the porous material is substantially free of the crystals of the pharmaceutically active species having a size of 1 micron or greater.

In some embodiments, the step of contacting comprises combining a solution comprising the pharmaceutically active species and a fluid carrier with the porous material. In some embodiments, the solution further comprises a surfactant. The solution may be, in some cases, in the form of droplets. In some embodiments, the step of contacting comprises exposure to ambient pressure. In some embodiments, the step of contacting comprises placing the porous material and pharmaceutically acceptable carrier under reduced pressure. In some embodiments, the step of contacting comprises heating the porous material and pharmaceutically acceptable carrier. In some embodiments, the step of contacting comprises cooling the porous material and pharmaceutically acceptable carrier. In some embodiments, the step of contacting comprises sonicating the porous material and pharmaceutically acceptable carrier.

In some embodiments, the set of conditions comprises removing at least a portion of the fluid carrier, or substantially all of the fluid carrier. In some embodiments, the set of conditions comprises adding a fluid carrier that facilitates formation of a crystal of the pharmaceutically active species.

In one set of embodiments, the method comprises combining a solution comprising the pharmaceutically active species and a fluid carrier with the porous material under ambient conditions such that the pharmaceutically active species enters the pores; filtering and/or washing the porous material containing the pharmaceutically active species within the pores; placing the porous material under the set of conditions which facilitates formation of a crystal of the pharmaceutically active species; and allowing the pharmaceutically active species to form the crystal within the plurality of pores.

In another set of embodiments, the method comprises combining a solution comprising the pharmaceutically active species and a fluid carrier with the porous material at a pressure greater than 1 atm such that the pharmaceutically active species enters the pores; filtering and/or washing the porous material containing the pharmaceutically active species within the pores; placing the porous material under the set of conditions which facilitates formation of a crystal of the pharmaceutically active species; and allowing the pharmaceutically active species to form the crystal within the plurality of pores.

In another set of embodiments, the method comprises combining a solution comprising the pharmaceutically active species and a fluid carrier with the porous material under reduced pressure such that the pharmaceutically active species enters the pores; filtering and/or washing the porous material containing the pharmaceutically active species within the pores; placing the porous material under the set of conditions which facilitates formation of a crystal of the pharmaceutically active species; and allowing the pharmaceutically active species to form the crystal within the plurality of pores.

In another set of embodiments, the method comprises sonicating a solution comprising the pharmaceutically active species and a fluid carrier and the porous material such that the pharmaceutically active species enters the pores; filtering and/or washing the porous material containing the pharmaceutically active species within the pores; placing the porous material under the set of conditions which facilitates formation of a crystal of the pharmaceutically active species; and allowing the pharmaceutically active species to form the crystal within the plurality of pores.

In any of the foregoing embodiments, the solution may further comprise a surfactant.

In any of the foregoing embodiments, the solution may be in the form of droplets.

In another set of embodiments, the method comprises combining the pharmaceutically active species in solid form with the porous material at a temperature at or above the melting temperature of the pharmaceutically active species and below the melting temperature of the porous material, such that the pharmaceutically active species enters the pores; cooling the porous material and pharmaceutically active species to facilitate formation of a crystal of the pharmaceutically active species; and filtering and/or washing the porous material containing the pharmaceutically active species within the pores.

In any of the foregoing embodiments, the method may further comprise applying centrifugal force to the porous material and pharmaceutically active species in order to remove gas (e.g., air), if present, within the pores. In any of the foregoing embodiments, the method may further comprise the step of compressing the porous material containing the pharmaceutically active species in crystal form into a tablet. In any of the foregoing embodiments, the method may further comprise the step of placing the porous material containing the pharmaceutically active species in crystal form within a capsule.

In any of the foregoing embodiments, the method may further comprise placing the porous material under a second set of conditions, which facilitates growth of the crystal of the pharmaceutically active species, in addition to (in one embodiment, after) formation of the crystal and growing the crystal of the pharmaceutically active species within the plurality of pores. In some embodiments, the second set of conditions facilitates spontaneous nucleation of the pharmaceutically active species in an amount less than about 10% (e.g., less than about 5%, less than about 1%), or essentially does not facilitate spontaneous nucleation of the pharmaceutically active species. In some embodiments, after the growth step, the exterior surface of the porous material is substantially free of crystals of the pharmaceutically active species having a size of 1 micron or greater. In some embodiments, the second set of conditions is different from the set of conditions. In some embodiments, the relative percent loading of pharmaceutically active species in the porous material after the growing step is greater than or equal to about 20%, greater than or equal to about 50%, or greater than or equal to about 70%. In some embodiments, the relative percent loading of the pharmaceutically active species in the porous material after the growing step is between about 30% and about 95% or between about 70% and about 90%.

In any of the foregoing embodiments, the method may be carried out as a batch, semi-batch, or continuous process.

Materials comprising a pharmaceutically active species are also provided. Some embodiments provide materials comprising a pharmaceutically active species, prepared by the method according to any of the foregoing embodiments. In some embodiments, the material comprising the pharmaceutically active species comprises a porous material comprising a plurality of pores having an average pore size of about 10 nm or greater; and a pharmaceutically active species in crystal form positioned within the plurality of pores.

Pharmaceutical compositions are also provided. In some embodiments, the pharmaceutical composition comprises a porous material comprising a plurality of pores; a pharmaceutically active species in crystal form positioned within the plurality of pores; and a pharmaceutically acceptable carrier.

In any of the foregoing embodiments, the exterior surface of the porous material may be substantially free of the crystals of the pharmaceutically active species having a size of 1 micron or greater.

In any of the foregoing embodiments, the porous material is a biologically compatible porous material. For example, in any of the foregoing embodiments, the porous material may comprise cellulose, cellulose acetate, carbon, silicon dioxide, titanium dioxide, aluminum oxide, other glass materials, or combinations thereof. In any of the foregoing embodiments, the porous material comprises a plurality of pores having an average pore size of about 10 nm or greater. In some embodiments, the plurality of pores has an average pore size in the range of about 10 nm to about 1000 nm, about 10 nm to about 500 nm, about 10 nm to about 250 nm, about 10 nm to about 100 nm, or about 30 nm to about 100 nm. In some embodiments, the plurality of pores has an average pore size in the range of about 30 nm to about 100 nm.

In any of the foregoing embodiments, the pharmaceutically active species is substantially insoluble or at least has low solubility in aqueous solutions, in the absence of association with the porous material. In some cases, in the absence of association with the porous material, the pharmaceutically active species when having a particle size greater than about 1000 nm has a solubility of less than 0.1 mg/mL in aqueous solution at room temperature. In some cases, the pharmaceutically active species may be ibuprofen, deferasirox, felodipine, griseofulvin, bicalutamide, glibenclamide, indomethacin, fenofibrate, itraconazole, or ezetimibe.

In any of the foregoing embodiments, the 80% dissolution of the pharmaceutically active species in crystal form within the pores occurs at least about 10% faster than that of the pharmaceutically active species in crystal form that is not within the pores and that has a particle size greater than about 1000 nm.

In any of the foregoing embodiments, the amount of the pharmaceutically active species in crystal form within the pores that is dissolved five minutes after contact with an aqueous solution is at least about 10% greater than that of the pharmaceutically active species in crystal form that is not within the pores and that has a particle size greater than about 1000 nm.

In any of the foregoing embodiments, the relative percent loading of the pharmaceutically active species in the porous material is greater than or equal to about 20%, greater than or equal to about 50%, or greater than or equal to about 70%. In some embodiments, the relative percent loading of the pharmaceutically active species in the porous material is between about 20% and about 80% or between about 70% and about 90%.

### BRIEF DESCRIPTION OF THE DRAWINGS

FIGS. 1A-1B show schematic representations of (1A) a porous material incorporating a pharmaceutically active species within its pores and (1B) porous materials comprising various types of pores including an open pore, a closed pore, and a tortuous pore network.

FIG. 2 shows (a) an image of droplets generated and dispensed by a Nano-Plotter® and (b) a schematic diagram of droplets dispensed onto the surface of a porous material.

FIG. 3 shows a graph of the dissolution tests of nano-crystalline ibuprofen loaded inside porous silicon dioxide particles (pore size of about 40 nm) compared to a physical mixture of crystalline ibuprofen and porous silicon dioxide particles.

FIG. 4 shows a graph of loading of ibuprofen nanocrystals within controlled pore glass (for the process described in example 8) with increasing solution concentration.

FIG. 5 shows an X-ray powder diffraction (XRPD) pattern of controlled pore glass (CPG) containing crystalline form I ibuprofen (IBP) compared to that of the theoretical pattern for form I IBP (CCDC refcode. IBPRAC02).

FIGS. 6A-6C show (6A) a scanning electron microscopy (SEM) image and (6B) a differential scanning calorimetry (DSC) thermogram of ibuprofen crystallized in CPG, and (6C) a graph showing the dissolution rates of ibuprofen crystallized in CPG and of 200 mg of the formulated tablet known as Advil®.

FIGS. 7A-7B show (7A) a DSC thermogram comparing the melting points of fenofibrate crystallized within the CPG with that of bulk-sized crystals (>2 μm) of fenofibrate and (7B) the dissolution rate of the fenofibrate crystallized in CPG compared to that of a TriCor tablet.

FIGS. 8A-8C show schematic representations of a continuous process for generating porous materials loaded with pharmaceutically active species in crystal form, involving (8A) washing the porous materials in a continuous stirred tank reactor; (8B) spray-washing the porous materials; and (8C) use of a rotating basket containing the porous materials.

FIG. 9 shows a schematic representation of a two stage process for generating porous materials loaded with pharmaceutically active species in crystal form and growing the pharmaceutically active species crystals within the pores.

FIGS. 10A-10C show X-ray powder diffraction (XRPD) patterns of (10A) bulk fenofibrate, (10B) fenofibrate loaded in the pores of 53 nm CPG, and (10C) fenofibrate loaded in the pores of CPG and AEROPERL.

FIG. 11 shows differential scanning calorimetry (DSC) scans of various CPGs containing fenofibrate loaded in the pores.

FIGS. 12A-12B show dissolution profiles of fenofibrate nanocrystals in various CPG differing in pore size, crushed bulk fenofibrate, and uncrushed bulk fenofibrate.

FIG. 13 shows dissolution profiles of nanocrystalline fenofibrate in AEROPERL compared to bulk crushed fenofibrate.

Other aspects, embodiments, and features of the invention will become apparent from the following detailed description when considered in conjunction with the accompanying drawings. The accompanying figures are schematic and are not intended to be drawn to scale. For purposes of clarity, not every component is labeled in every figure, nor is every component of each embodiment of the invention shown where illustration is not necessary to allow those of ordinary skill in the art to understand the invention. All patent applications and patents incorporated herein by reference are incorporated by reference in their entirety. In case of conflict, the present specification, including definitions, will control.

### DETAILED DESCRIPTION

Materials and methods related to pharmaceutically active species in solid (e.g., crystal) form are provided. In some cases, the material may include a pharmaceutically active species associated with a porous support material, and the material may be administered as a pharmaceutical product. Some embodiments described herein allow for improved stability, solubility, bioavailability, and/or dissolution rates for pharmaceutically active species having poor aqueous solubility (e.g., in the absence of a porous support material). In some cases, the method may involve the loading and subsequent crystallization of pharmaceutically active species within pores (e.g., nanopores) of a porous support material, such as a porous excipient material. This may eliminate the need for additional excipient materials, co-solvents, surfactants, and other additives that can have adverse effects on a subject in vivo. Such materials can simplify both the production and formulation of nanosized active pharmaceutical ingredients.

The materials described herein may advantageously contain crystalline forms of a pharmaceutically active species, rather than amorphous forms. This may result in pharmaceutical products with improved chemical and/or physical stability since amorphous forms of pharmaceutically active species can often convert to crystalline forms during storage, resulting in inconsistencies in dissolution rate and/or performance. By contrast, crystalline forms of pharmaceutically active species are relatively stable and, when arranged within porous materials as described herein, can produce pharmaceutical products with improved performance. In some cases, the materials described herein include crystals of a single pharmaceutically active species or, alternatively, multi-component crystals such as salts and/or co-crystals of pharmaceutically active species.

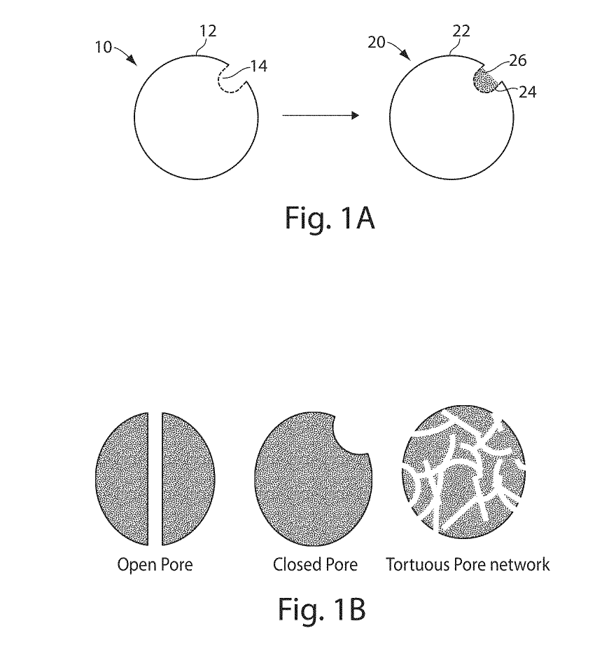

Another advantageous feature of embodiments described herein is the ability to form materials containing a pharmaceutically active species arranged within an interior portion of the material (e.g., within a pore), while the exterior surface of the material (e.g., surfaces that are not within pores) may be substantially free of the pharmaceutically active species. For example, the exterior surface of the porous material may be substantially free of bulk-sized crystals of the pharmaceutically active species, i.e., crystals of the pharmaceutically active species having a particle size of 1 micron or greater. For example, FIG. 1A shows a schematic representation of a porous material 10, which includes an exterior (e.g., non-pore) surface 12 and an interior pore surface 14. Embodiments described herein may provide materials where a pharmaceutically active species may be formed or arranged within the pores, while the exterior surface (e.g., non-interior pore surface) of the material may be substantially free of the crystals of the pharmaceutically active species having a size of 1 micron or greater. As shown in FIG. 1A, porous material 20 includes pharmaceutically active species 26 in solid form arranged within a pore such that pharmaceutically active species 26 contacts pore surface 24 but substantially does not contact exterior surface 22.

As used herein, a surface that is “substantially free” of crystals of the pharmaceutically active species having a size of 1 micron or greater refers to a surface that contains less than 10% (relative to the total surface area) of pharmaceutically active species crystals having a size of 1 micron or greater, as determined by SEM. In some cases, the porous material has an exterior surface that contains less than 10% (relative to the total exterior surface area) of pharmaceutically active species crystals having a size of 1 micron or greater. In some cases, the porous material has an exterior surface that contains about 10%, about 8%, about 6%, about 4%, about 2%, about 1%, or less than about 1% (relative to the total exterior surface area) of pharmaceutically active species crystals having a size of 1 micron or greater.

In some cases, incorporation of the pharmaceutically active species within a porous material may advantageously affect certain properties of the pharmaceutically active species. For example, the ability to contain, and form crystals of, the pharmaceutically active species within relatively small pores may increase the solubility, dissolution rate, and/or bioavailability of the pharmaceutically active species, relative to the same pharmaceutically active species (and crystals thereof) not contained within a porous material. In some cases, the ability to form crystals having relatively smaller particle sizes (e.g., the size of the pores) may increase the solubility of the pharmaceutically active species. This may be attributed at least in part to the larger surface-to-volume ratios provided by such nanosized particles or crystals. In some cases, particles of pharmaceutically active species (e.g., within the pores) may have an average particle size in the nanometer range (e.g., less than 1000 nm). The presence of a crystal form of a solid may be evaluated using methods known in the art, such as X-ray diffraction (e.g., X-ray powder diffraction) and differential scanning calorimetry.

Generally, solubility increases as particle size of a pharmaceutically active species decreases. In some embodiments, the pharmaceutically active species in crystal form within the pores (e.g., for average particle sizes of approximately 20˜1000 nm) has a solubility that is at least about 10% greater than that of the pharmaceutically active species in crystal form that is not within the pores and that has a particle size greater than about 1000 nm. For example, the pharmaceutically active species in crystal form within the pores may have a solubility about 10%, about 20%, about 30%, about 40%, about 50%, about 60%, about 70%, about 80%, or about 90% greater than that of the pharmaceutically active species in crystal form that is not within the pores and that has a particle size greater than about 1000 nm. In some cases, the pharmaceutically active species in crystal form within the pores (e.g., for average particle sizes of approximately 20˜1000 nm) has a solubility that is about 2, about 5, about 10, about 20, about 30, about 40, or about 50 times greater than that of the pharmaceutically active species in crystal form that is not within the pores and that has a particle size greater than about 1000 nm.

Typically, dissolution rate of small crystals increase in proportion to the increase in both surface area and solubility of the pharmaceutically active species. However, the dissolution rate of the pharmaceutically active species in crystal form within the pores may also be affected by diffusion. In some embodiments, the pharmaceutically active species in crystal form within the pores (e.g., for average particle sizes of approximately 20˜1000 nm) has a dissolution rate that is at least about 10% greater than that of the pharmaceutically active species in bulk crystal form, i.e., a crystal that is not within the pores and that has a particle size greater than about 1000 nm (1 micron). In some embodiments, the dissolution rate refers to the amount of time in which 80% of the pharmaceutically active species is dissolved in an aqueous solution. For example, the pharmaceutically active species in crystal form within the pores may have a dissolution rate that is about 20%, about 30%, about 40%, about 50%, about 60%, about 70%, about 80%, about 90%, about 95%, about 100%, or, in some cases, about 200%, about 300%, about 400%, about 500%, about 600%, about 700%, about 800%, about 900%, or in some cases, about 1000% greater than that of the pharmaceutically active species in crystal form that is not within the pores and that has a particle size greater than about 1000 nm. In some embodiments, the pharmaceutically active species in crystal form within the pores (e.g., for average particle sizes of approximately 20 - 1000 nm) has a dissolution rate that is about 10, about 50, about 100, about 250, about 500, about 750, about 1000, about 1500, or about 2000 times greater than that of the pharmaceutically active species in crystal form that is not within the pores and that has a particle size greater than about 1000 nm.

In some embodiments, 80% dissolution of the pharmaceutically active species in crystal form within the pores occurs at least about 10% faster or at least 20% faster, than 80% dissolution of the pharmaceutically active species in crystal form that is not within the pores and that has a particle size greater than about 1000 nm. In some embodiments, 80% dissolution of the pharmaceutically active species in crystal form within the pores occurs at least about 30%, about 40%, about 50%, about 60%, about 70%, about 80%, about 90%, about 95%, about 100%, or, in some cases, about 200%, about 300%, about 400%, about 500%, about 600%, about 700%, about 800%, about 900%, or in some cases, about 1000% faster than 80% dissolution of the pharmaceutically active species in crystal form that is not within the pores and that has a particle size greater than about 1000 nm.

In some embodiments, the amount of pharmaceutically active species in crystal form within the pores that is dissolved five minutes after contact with an aqueous solution is at least about 10% greater than that of the pharmaceutically active species in crystal form that is not within the pores and that has a particle size greater than about 1000 nm. In some embodiments, the amount of pharmaceutically active species in crystal form within the pores that is dissolved five minutes after contact with an aqueous solution is about 10%, about 20%, about 30%, about 40%, about 50%, about 60%, about 70%, about 80%, about 90%, about 95%, about 100%, or, in some cases, about 200%, about 300%, about 400%, about 500%, about 600%, about 700%, about 800%, about 900%, or in some cases, about 1000% greater than that of the pharmaceutically active species in crystal form that is not within the pores and that has a particle size greater than about 1000 nm (1 micron).

In some embodiments, the melting point of the pharmaceutically active species may be reduced upon incorporation within a porous material. In some embodiments, bioavailability of the pharmaceutically active species may be enhanced upon incorporation within a porous material.

In some cases, methods for preparing such materials are provided. The method may involve impregnating or loading a porous material with a pharmaceutically active species using various methods. For example, the pharmaceutically active species (e.g., in solution, or in solid form) may be brought into contact with the porous material under conditions which allow the pharmaceutically active species to enter the pores of the porous material. In some embodiments, the pharmaceutically active species is provided in solid form. In some embodiments, the pharmaceutically active species is combined with a fluid carrier (e.g., solvent). In some embodiments, the pharmaceutically active species is provided in solution form. For example, the solution may contain the pharmaceutically active species, a solvent or fluid carrier, and optionally other species (e.g., such as surfactants) that may facilitate solubility of the pharmaceutically active species in the solution, penetration of the solution within the pores of the porous material, and/or may otherwise improve formation of the materials. In one set of embodiments, the solution may be in the form of droplets.

The solution may contain an amount of the pharmaceutically active species that is below the level at which crystallization or precipitation of the pharmaceutically active species occurs (e.g., under saturation levels). In other cases, it may be desirable to contact the porous material with a solution containing the pharmaceutically active species at, around, or above the level at which crystallization or precipitation of the pharmaceutically active species occurs (e.g., saturation or super-saturation levels). The porous material loaded with the pharmaceutically active species may then be separated from the solution, via filtering, washing, and/or other methods, and, optionally, may be dried (e.g., under ambient conditions, under reduced pressure, by heating, etc.).

In some embodiments, a solution containing the pharmaceutically active species and a fluid carrier may be combined with the porous material. The solution and porous material may be combined under ambient conditions (e.g., ambient temperature and/or ambient pressure) and for a sufficient time period such that the pharmaceutically active species can enter the pores via diffusion/equilibration. In some cases, the solution containing the pharmaceutically active species and a fluid carrier may be combined with the porous material and placed under increased pressure. In some cases, the solution containing the pharmaceutically active species and a fluid carrier may be combined with the porous material and placed under reduced pressure. In some embodiments, the solution containing the pharmaceutically active species may be in the form of droplets and may be sprayed or otherwise applied to the porous material. For example, solution droplets can be generated and dispensed onto the surface of a porous material, where the droplets enter the pores via capillary action. In some cases, it may be desirable to heat the solution containing the pharmaceutically active species and/or the porous material to a temperature greater than about 25° C. In some cases, it may be desirable to cool the solution containing the pharmaceutically active species and/or the porous material to a temperature less than about 25° C.

The solution and/or porous material may be treated (e.g., sonicated, degassed, centrifuged, etc.) in order to remove or reduce the amount of gas (e.g., oxygen) within the porous material, facilitating entry of the pharmaceutically active species into the pores. In some cases, the solution containing the pharmaceutically active species and the fluid carrier may be combined with the porous material, and the mixture may be sonicated. In some cases, the solution containing the pharmaceutically active species may be combined with the porous material, and the mixture may be degassed. In some cases, the solution containing the pharmaceutically active species and the fluid carrier may be combined with the porous material, and the mixture may be centrifuged. In some cases, the pharmaceutically active species in solid form may be combined with the porous material and heated above the melting temperature of the pharmaceutically active species, but below the melting temperature of the porous material. The melted pharmaceutically active species, in liquid form, may then enter the pores via, for example, capillary action. The loaded porous material may then be cooled and separated, washed, and/or filtered from the excess amount of pharmaceutically active species.

Any of the described embodiments for introducing the pharmaceutically active species within the porous material may be utilized alone or in combination. For example, centrifugal force may be applied to a mixture containing the pharmaceutically active species, the porous material, and a fluid carrier, followed by sonication/degassing at reduced temperature in order to facilitate entry of the pharmaceutically active species into the pores.

Upon loading onto the porous material, the pharmaceutically active species may then be placed under a set of conditions which promotes formation of a solid form (e.g., crystal form) of the pharmaceutically active species. In some cases, the solid form may be a crystal, including specific polymorphs of a crystal. In some cases, the solid form may be amorphous. In some embodiments, the solid form of the pharmaceutically active species may be substantially contained within the pores of the porous material, i.e., the exterior surface of the porous material may be substantially free of crystals of the pharmaceutically active species having a size of 1 micron or greater.

As used herein, a “set of conditions” or “conditions” may comprise, for example, a particular temperature, pH, solvent, chemical reagent, type of atmosphere (e.g., nitrogen, argon, oxygen, etc.), electromagnetic radiation, or the like. Some embodiments may involve a set of conditions comprising exposure to a source of external energy. The source of energy may comprise electromagnetic radiation, electrical energy, sound energy, thermal energy, or chemical energy. For example, the set of conditions may involve exposure to heat or electromagnetic radiation. In some embodiments, the set of conditions includes exposure to a particular temperature or pH.

In some cases, the set of conditions may be selected to facilitate crystallization of the pharmaceutically active species within the pores. For example, the set of conditions may involve removal of at least a portion of the fluid carrier in order to bring the solution to saturation levels that facilitate crystallization (i.e., to cause super-saturation). In some cases, substantially all of the fluid carrier may be removed. In another set of embodiments, a fluid carrier that facilitates formation of a crystal (e.g., a non-solvent) may be added to the pharmaceutically active species. The set of conditions may also involve heating and/or cooling the pharmaceutically active species within the porous material, and the fluid carrier. Those of ordinary skill in the art would be capable of selecting the appropriate conditions in order to promote formation of a crystal.

In one set of embodiments, the porous material loaded with the pharmaceutically active species may be formed by mixing the porous material with a solution containing the pharmaceutically active species and a fluid carrier. In some cases, the fluid carrier may be solvent in which the pharmaceutically active species is substantially soluble. For example, the pharmaceutically active species may be dissolved in a solvent to form a solution, which is then combined with a porous material (e.g., nanoporous material) as described herein for a sufficient time period such that the solution may penetrate and/or enter pores of the porous material (e.g., by diffusion/equilibration). In some cases, the porous material and the solution containing the pharmaceutically active species are combined under ambient conditions. The loaded or impregnated porous material may then be separated from excess solution by filtration and washed to substantially remove solution or any pharmaceutically active species from the exterior surface of the porous material. Thereafter, crystallization/precipitation of the pharmaceutically active species within the pores may be induced using techniques to supersaturate the solution within the pores containing the pharmaceutically active species, such as cooling, addition of an anti-solvent, or evaporation. In some cases, washing excess solution from the exterior surface of the porous material prior to crystallization/precipitation of the pharmaceutically active species may reduce or prevent formation of crystals (e.g., bulk-sized crystals) on the exterior surface.

In another set of embodiments, the pharmaceutically active species may be dissolved in a solvent to form a solution, which is then combined with a porous material as described herein at a pressure greater than 1 atm such that the solution may penetrate and/or enter pores of the porous material. In some cases, the pressure may be in the MPa range and maintained for a sufficient time period to allow for impregnation of the pharmaceutically active species solution within the porous material. The pressure may then be reduced to allow for separation and filtration of the impregnated porous material from excess solution, followed by washing. Crystallization/precipitation of the pharmaceutically active species within the pores may then be induced as described herein.

In some cases, the solution containing the pharmaceutically active species and a fluid carrier may be combined with the porous material and placed under reduced pressure. For example, the solution containing the pharmaceutically active species may be placed within a container topped with a lid having a plurality of perforations, and the container may be placed lid-down in a larger vessel capable of being placed under reduced pressure. The solution containing the pharmaceutically active species may be introduced into the vessel until atmospheric pressure has been reached or until a sufficient amount of the pharmaceutically active species have entered the pores. The loaded or impregnated porous material may then be separated from excess solution by filtration and washed to substantially remove solution or any pharmaceutically active species from the exterior surface of the porous material. Crystallization/precipitation of the pharmaceutically active species within the pores may be induced as described herein.

Methods disclosed herein may be performed as a batch, semi-batch, or continuous process. In semi-batch processes, a portion of the process is performed as a batch process and another portion of the process is performed as a continuous process.

In some embodiments, methods disclosed herein may be performed as a continuous process. For example, one or more steps of the method may be conducted within a continuous stirred tank reactor (CSTR), reaction/separation columns, continuous crystallizers, filter belts, fluidized bed dryers, and the like. The porous material may be contacted with a pharmaceutically active species in solution, as a neat liquid melt, as a sublimed vapor, or the like, as described herein, followed by various steps to produce the final material, including filtration, rinsing or washing, heating/cooling, evaporation of solvent, and/or crystallization.

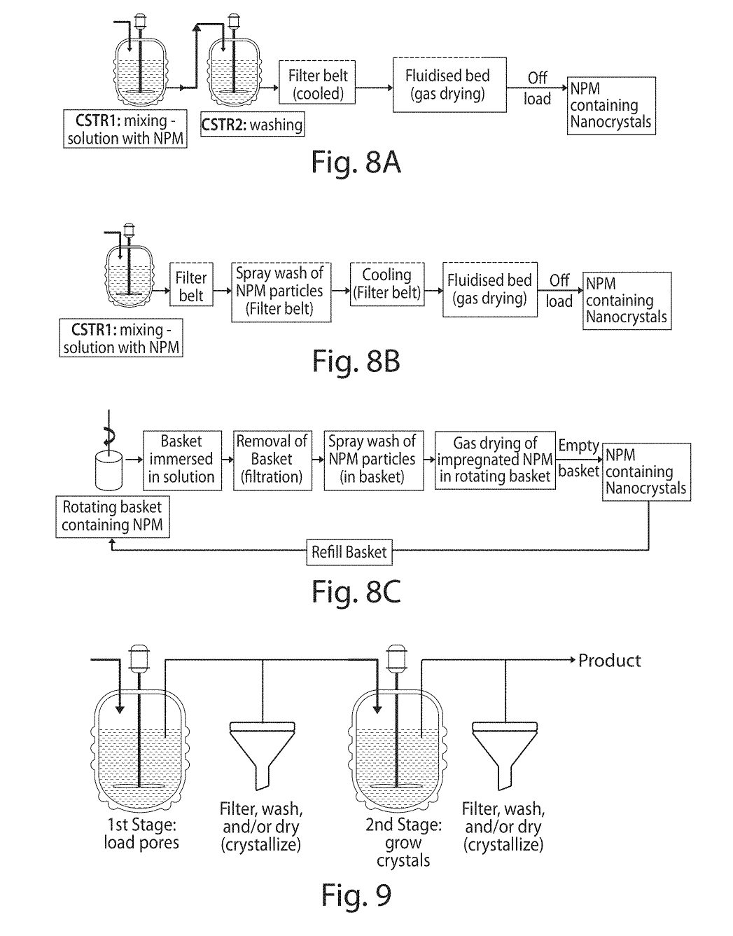

FIG. 8A shows an illustrative embodiment for a continuous process, involving mixing a porous material (e.g., nanoporous material or NPM) with a solution of pharmaceutically active species in a first continuous stirred tank reactor, followed by washing the impregnated porous material in a second continuous stirred tank reactor. Upon subsequent cooling, and drying on a fluidized bed, the porous material containing the pharmaceutically active species in crystal form may be recovered. FIG. 8B shows another embodiment where a porous material (e.g., nanoporous material or NPM) is mixed with a solution of pharmaceutically active species in a continuous stirred tank reactor, followed by spray-washing to remove excess solution/pharmaceutically active species. Subsequent cooling, and drying on a fluidized bed, is performed in order to produce the final material. FIG. 8C illustrates an embodiment involving a rotating basket containing a porous material (e.g., nanoporous material or NPM), which is submerged in a solution comprising pharmaceutically active species. Upon removal of the basket from the solution, the resulting impregnated porous material may be subsequently washed (e.g., spray-washed) and dried to produce the final material. Those of ordinary skill in the art would be capable of selecting appropriate reaction vessels and other equipment to suit a particular continuous process.

In some embodiments, in addition to (in one embodiment, after) formation of crystals of a pharmaceutically active species using the methods described above, the porous material may be subjected to one or more processing steps. In certain embodiments, the porous material may be subjected to a process designed to increase the loading of the pharmaceutically active species. For instance, the porous material comprising a plurality of pores containing crystals of a pharmaceutically active species may be subjected to a crystal growth process, in which crystals in the pores of the porous material serve as seed crystals. The crystal growth process may comprise placing the porous material under a set of conditions, which facilitates growth of the crystal of the pharmaceutically active species and allowing the crystals to grow or otherwise increase in size and/or mass. In some such cases, the set of conditions facilitates spontaneous nucleation of the pharmaceutically active species in an amount less than about 10% (e.g., less than about 5%, less than about 1%), or essentially does not facilitate spontaneous nucleation of the pharmaceutically active species, such that the increase in mass is not attributed to the formation of crystals on an exterior surface. The increase in mass may be attributed to the growth of crystals in pores of the porous material. In some embodiments, after the crystal growth process, the exterior surface of the porous material may be substantially free of bulk-sized crystals of the pharmaceutically active species, i.e., crystals of the pharmaceutically active species having a particle size of 1 micron or greater.

In some embodiments, the crystal growth process may be distinct from the crystal growth that occurs as part of the crystallization process in the previous step, described above. For instance, crystal growth may occur under a different set of conditions than the crystallization process (e.g., crystal formation) and/or one or more intervening process (e.g., filtration, drying, washing) may occur between crystallization and the crystal growth process. In one example, a method for loading and/or forming a solid (e.g., crystalline) pharmaceutically active species within pores of a porous material may comprise crystallizing a pharmaceutically active species within pores of a porous material under a first set of conditions to form crystals of the pharmaceutically active species within the pores and growing the crystals under a second set of conditions, wherein, upon formation of the crystals within the plurality of the pores and/or after the growth step, the exterior surface of the porous material may be substantially free of crystals of the pharmaceutically active species having a size of 1 micron or greater. The second set of conditions may be different from the first set of conditions.

As another example, a method for increasing the mass of a solid (e.g., crystalline) pharmaceutically active species within pores of a porous material may comprise contacting a porous material comprising crystals of a pharmaceutically active species within a plurality of pores with a solution comprising the pharmaceutically active species (e.g., supersaturated solution of the pharmaceutically active species), such that the pharmaceutically active species enters the pores. The exterior surface of the porous material may be substantially free of crystals of the pharmaceutically active species having a size of 1 micron or greater. The mass of solid pharmaceutically active species within pores of a porous material may be increased by growing the crystals under a set of conditions that facilitates crystal growth and/or facilitates spontaneous nucleation of the pharmaceutically active species in an amount less than about 10% (e.g., less than about 5%, less than about 1%), or essentially does not facilitate spontaneous nucleation of the pharmaceutically active species (e.g., on the exterior surface). After crystal growth, the exterior surface of the porous material may be substantially free of crystals of the pharmaceutically active species having a size of 1 micron or greater. Prior to the contacting step (e.g., after crystal formation), the porous material may be filtered, dried, and/or washed.

In general, the weight percentage of pharmaceutically active species in the porous material after the crystal growth process is greater than the weight percentage prior to the crystal growth process (e.g., after the crystal formation). In some embodiments, the relative percent loading may significantly increase after the crystal growth process. For example, the relative percent loading of the pharmaceutically active species in a porous material may be greater than or equal to about 20% and less than about 70% prior to a crystal growth process (e.g., after crystallization) and may be greater than or equal to about 70% and less than about 95% after the crystal growth process. As used herein, the relative percent loading may refer to the actual total mass of crystalline pharmaceutically active species in the pores of the porous material divided by the theoretical maximum mass of the same crystalline pharmaceutically active species in the pores of the porous material multiplied by 100. One of ordinary skill in the art would be able to calculate the theoretical maximum mass based on the total pore volume of the porous material, mass of the porous material before and after loading, and density of the crystalline pharmaceutically active species.

In some embodiment, the relative percent loading after a crystal growth process, as described herein, may be relatively high. For instance, in some embodiments, the relative percent loading after crystal formation (e.g., crystallization) may be greater than or equal to about 40%, greater than or equal to about 45%, greater than or equal to about 50%, greater than or equal to about 55%, greater than or equal to about 60%, greater than or equal to about 65%, greater than or equal to about 70%, greater than or equal to about 75%, greater than or equal to about 80%, or greater than or equal to about 85% and, in some instances, less than about 95%. In certain embodiments, the relative percent loading after crystal formation (e.g., crystallization) may be between about 30% and about 95%, between about 35% and about 95%, between about 40% and about 95%, between about 45% and about 95%, between about 50% and about 95%, between about 60% and about 95%, between about 70% and about 95%, or between about 70% and about 90%.

In some embodiments, the relative percent loading after the crystal formation step (e.g., crystallization) may be less than the yield after a crystal growth process. For instance, in some embodiments, the relative percent loading after crystal formation may be greater than or equal to about 20%, greater than or equal to about 25%, greater than or equal to about 30%, greater than or equal to about 35%, greater than or equal to about 40%, greater than or equal to about 45%, greater than or equal to about 50%, greater than or equal to about 55%, greater than or equal to about 60%, or greater than or equal to about 65% and, in some instances, less than about 70%. In certain embodiments, the relative percent loading after crystal formation may be between about 20% and about 70%, between about equal to about 20% and about 60%, between about 20% and about 50%, or between about 20% and about 40%. As mentioned above, the crystal growth process may be performed under a set of conditions that facilitates crystal growth within the pores. For example, the set of conditions may involve immersing and/or incubating the porous material containing crystals of pharmaceutically active species in a solution super-saturated with pharmaceutically active species. In some such cases, the super-saturation level is not within the metastable zone necessary for spontaneous nucleation of the pharmaceutically active species and/or facilitates spontaneous nucleation of the pharmaceutically active species in an amount less than about 10% (e.g., less than about 5%, less than about 1%), or essentially does not facilitate spontaneous nucleation of the pharmaceutically active species. In another set of embodiments, a material that facilitates growth of a crystal (e.g., a non-solvent, anti-solvents, surfactants) may be added to the solution. The set of conditions may also involve heating and/or cooling the pharmaceutically active species within the porous material, and the fluid carrier. Those of ordinary skill in the art would be capable of selecting the appropriate conditions in order to promote crystal growth.

In certain embodiments, in addition to (in one embodiment, prior to) crystal growth (e.g., immediately prior to crystal growth), the pharmaceutically active species may be brought into contact with the porous material under conditions which allow the pharmaceutically active species to enter the pores of the porous material containing crystals of the pharmaceutically active species. In some such cases embodiments, one or more of the loading conditions (e.g., pharmaceutically active species form, temperature, pressure, time) and/or methods utilized to facilitate entry of the pharmaceutically active species described above with respect to crystal formation may be used in the crystal growth process. For example, the pharmaceutically active species may be provided in solution form and the solution may contain the pharmaceutically active species, a solvent or fluid carrier, and optionally other species (e.g., such as surfactants) that may facilitate solubility of the pharmaceutically active species in the solution, penetration of the solution within the pores of the porous material, and/or may otherwise improve growth of the materials. In some embodiments, the solution may be super-saturated with pharmaceutically active species. In some such cases, the super-saturation level does not facilitate spontaneous nucleation of the pharmaceutically active species. In other embodiments, saturation or under-saturation levels may be used to load pharmaceutically active species into the pore of the porous material.

It should be understood that as used herein, crystal growth has its ordinary meaning in the art and may refer to the process by which an atom or molecule of the same chemical composition as the crystal is deposited on a surface of the crystal, such that addition of the new material does not substantially change the overall crystal structure. In general, crystal growth may consists of one or more transport steps (e.g., transport of atoms or molecules through a fluid) and one or more surface steps (e.g., attachment of the atoms or molecules to the crystal surface, movement of the atoms on the surface, and attachment of atoms or molecules to edges and kinks).

It should also be understood that as used herein crystal formation may refer to crystallization, which includes nucleation and initial crystal growth.

The crystal growth process disclosed herein may be performed as a batch, semi-batch, or continuous process. In some embodiments, the crystal growth process disclosed herein may be performed as a continuous process. For example, one or more steps of the method may be conducted within a mixed suspension mixed product removal (MSMPR) device, a continuous stirred tank reactor, plug flow reactor, tubular crystallizer, oscillatory baffled reactor, T-mixed reactor, a fluidized bed, and the like. In some embodiments, the crystallization process may be a stage in a manufacturing process configured to crystallize a pharmaceutically active species within pores of a porous material, such that the porous material has a certain weight percentage or relative percent loading of the pharmaceutically active species. In some such cases, the process may comprise a first stage for crystallization and a second stage for further crystal growth. In some instances, one or more stages (e.g., crystallization stage and crystal growth stage) may comprise contacting the porous material with a pharmaceutically active species in solution, as a neat liquid melt, as a sublimed vapor, or the like, as described herein, followed by various steps to produce a product for a subsequent stage or the final product, including filtration, rinsing or washing, heating/cooling, and/or evaporation of solvent.

FIG. 9 shows an illustrative embodiment for a two-stage continuous process, involving crystallization and crystal growth. In some embodiments, as shown in FIG. 9, the first stage may be a crystallization stage and may comprise one or more of the processes described above with respect to FIGS. 8A-8C. In certain embodiments, the first stage may be performed in a mixed suspension mixed product removal device. The first stage may comprise mixing a porous material with a solution of pharmaceutically active species in a mixed suspension mixed product removal device to load pharmaceutically active species within pores of the porous material. The porous material may then be removed from the mixed suspension mixed product removal device (e.g., via filtration), optionally washed and/or dried, and subjected to a first set of conditions that facilitates crystallization. After crystallization, the porous material containing crystals of the pharmaceutically active species may optionally be subjected to one or more intervening process (e.g., washing, drying). Regardless of the process used in the first stage, the second stage may comprise mixing the porous material containing crystals of the pharmaceutically active species with a solution of pharmaceutically active species in a device (e.g., a mixed suspension mixed product removal device) under a second set of conditions that facilitates crystal growth. The porous material containing crystalline pharmaceutically active species may be recovered (e.g., via filtration) and subsequent processing (e.g., drying) may be performed in order to produce a product for a subsequent stage or the final product.

The pharmaceutically active species (e.g., in crystal form) may have an average particle size that correlates to the average pore size of the porous material within which the pharmaceutically active species is formed or contained. In some embodiments, the average particle size of the pharmaceutically active species (in crystal form) within the porous material is about 10 nm or greater, about 20 nm or greater, about 30 nm or greater, about 40 nm or greater, or, in some cases, 50 nm or greater. In some cases, the average particle size of the pharmaceutically active species (in crystal form) within the porous material is in the range of about 10 nm to about 1000 nm, about 10 nm to about 500 nm, about 10 nm to about 250 nm, about 10 nm to about 100 nm, or about 30 nm to about 100 nm. In one set of embodiments, the average particle size of the pharmaceutically active species (in crystal form) within the porous material is in the range of about 40 nm to about 100 nm. Particle size may be determined using SEM imaging of cross-sections of materials which may be cut by Cryo-Microtome. For materials which cannot be cut, average particle size can be inferred from measurable property changes and the knowledge that the crystal cannot be larger than the pore dimensions of the porous material.

The loaded or impregnated porous material, i.e., the porous material containing the pharmaceutically active species in solid form within its pores, may be further processed into, or incorporated within, various articles. In some cases, the loaded porous material may be processed into an article useful as a pharmaceutical or drug product. For example, the loaded porous material may be in powder form, granular form, in bead form, or another solid form, and may be compressed, molded, or otherwise processed to produce a tablet. In some embodiments, a mixture containing the loaded porous material and a pharmaceutically acceptable carrier or pharmaceutically acceptable diluent may be compressed and/or molded to form a tablet. In some cases, the loaded porous material may be incorporated within a capsule.

Some embodiments provide materials prepared using any of the methods described herein. Pharmaceutical compositions including the loaded porous materials described herein are also provided. In some embodiments, the pharmaceutical composition includes a porous material, a pharmaceutically active species, and a pharmaceutically acceptable carrier. The pharmaceutically active species may be in crystal form and may be positioned within the plurality of pores such that the exterior surface of the porous material is substantially free of crystals of the pharmaceutically active species having a size of 1 micron or greater.

The pharmaceutically active species may be any substance that is useful for therapy (e.g., human therapy, veterinary therapy), including prophylactic and therapeutic treatment. In some embodiments, the pharmaceutically active species may be a substance used as a medicine for treatment, prevention, delay, reduction or amelioration of a disease, condition, or disorder. In some embodiments, the pharmaceutically active species may enhance (e.g., increase) the effect or effectiveness of a second species, for example, by enhancing potency or reducing adverse effects of a second species. Pharmaceutically active species include organic molecules that are drug compounds, small molecules, peptides, proteins, carbohydrates, monosaccharides, oligosaccharides, polysaccharides, nucleoproteins, mucoproteins, lipoproteins, synthetic polypeptides or proteins, small molecules linked to a protein, glycoproteins, steroids, nucleic acids, DNA molecules, RNA molecules, nucleotides, nucleosides, oligonucleotides, antisense oligonucleotides, lipids, hormones, vitamins, and the like.

In some embodiments, the pharmaceutically active species is substantially insoluble, or at least has low solubility, in aqueous solutions (e.g., water, aqueous solutions containing water and a surfactant, etc.). For example, the pharmaceutically active species may have a solubility of less than 0.1 mg/mL in aqueous solutions (e.g., water) at room temperature, in the absence of being incorporated within a porous material (e.g., when the pharmaceutically active species has a particle size greater than about 1000 nm and is not positioned within pores of a porous material and). In some cases, the pharmaceutically active species may have an aqueous solubility of about 0.05 mg/mL or less, about 0.005 mg/mL or less, about 0.0005 mg/mL or less, about 0.00005 mg/mL or less, or about 0.000005 mg/mL or less, in the absence of being incorporated within a porous material. In some cases, the pharmaceutically active species may have an aqueous solubility in the range of about 0.000001 mg/mL to about 0.1 mg/mL, about 0.00001 mg/mL to about 0.1 mg/mL, about 0.0001 mg/mL to about 0.1 mg/mL, about 0.001 mg/mL to about 0.1 mg/mL, or about 0.01 mg/mL to about 0.1 mg/mL, in the absence of being incorporated within a porous material.

In some embodiments, the pharmaceutically active species is ibuprofen (aqueous solubility of 0.038 mg/mL), deferasirox (aqueous solubility of 0.038 mg/mL), felodipine (aqueous solubility of 0.019 mg/mL), griseofulvin (aqueous solubility of 0.00864 mg/mL), bicalutamide (aqueous solubility of 0.005 mg/mL), glibenclamide (aqueous solubility of 0.004 mg/mL), indomethacin (aqueous solubility of 0.0025 mg/mL), fenofibrate (aqueous solubility of 0.0008 mg/mL), itraconazole (aqueous solubility of 0.000001 mg/mL), or ezetimibe (essentially insoluble in aqueous solutions). It should be understood that these pharmaceutically active species are discussed by way of example only, and any pharmaceutically active species that is substantially insoluble in aqueous solutions, in the absence of association with the porous material, can be utilized within the context of embodiments described herein. Those of ordinary skill in the art would be capable of identifying such pharmaceutically active species (e.g., by identifying aqueous solubility value of the species, by combining a small amount of the species with an aqueous solution and observing the results, etc.).

Any of the embodiments described herein may include an effective amount of the pharmaceutically active species to achieve a desired therapeutic and/or prophylactic effect. In some embodiments, an effective amount of the pharmaceutically active species is at least a minimal amount of a species, or a composition containing a species, which is sufficient for treating one or more symptoms of a disorder or condition.

The porous material may be any material that contains various pores within which a pharmaceutically active species may be formed. In some cases, a non-porous material may be processed to include a plurality of pores to render it suitable for use in embodiments described herein. Generally, the porous material may be a biologically compatible material, or another material that can be used as an excipient for a pharmaceutically active species. The porous material may be, for example, a polymeric material. In some cases, the porous material may comprise an organic material. In some cases, the porous material may consist of an organic material. In some cases, the porous material may consist essentially of an organic material. In some cases, the porous material may comprise an inorganic material. In some cases, the porous material may consist of an inorganic material. In some cases, the porous material may consist essentially of an inorganic material. The porous material may include materials which are substantially soluble in aqueous solutions.

Examples of porous materials, or non-porous materials that may be processed into porous materials, include, but are not limited to, starches (e.g., corn starch, potato starch, pre-gelatinized starch, or others), gelatin, natural and synthetic gums (e.g., acacia, sodium alginate, alginic acid, other alginates, powdered tragacanth, guar gum), lactose including hydrates thereof (e.g., lactose monohydrate), dextrin, dextrates, cellulose and its derivatives (e.g., ethyl cellulose, hydroxyethyl cellulose, cellulose acetate, carboxymethyl cellulose calcium, sodium carboxymethyl cellulose, methyl cellulose, hydroxypropyl methyl cellulose, microcrystalline cellulose), polyvinyl pyrrolidone (or povidone), polyethylene oxide, polydextrose, polyoxamer, metal carbonates (e.g., magnesium carbonate) metal oxides (e.g., silicon dioxide, titanium dioxide, aluminum oxide, etc.), other glass materials, mixtures thereof, and the like. In some cases, the porous material comprises cellulose, cellulose acetate, carbon, silicon dioxide, titanium dioxide, aluminum oxide, other glass materials, or combinations thereof. In one set of embodiments, the porous material comprises cellulose. In one set of embodiments, the porous material comprises silicon dioxide.

The porous material may include one or more different types of pores. The pores may have different dimensions, cross-sectional shapes, and the like. FIG. 1B illustrates exemplary pores, including open pores, closed pores, and networks of pores.

In some cases, the porous material may comprise a plurality of nanopores, i.e., pores having an average pore size less than about 1000 nm but greater than about 1 nm. Some embodiments involve a porous material having a plurality of pores with an average pore size of about 10 nm or greater, or, in some cases, 40 nm or greater. In some cases, the plurality of pores may have an average pore size in the range of about 10 nm to about 1000 nm, about 10 nm to about 500 nm, about 10 nm to about 250 nm, about 10 nm to about 100 nm, or about 40 nm to about 100 nm. In one set of embodiments, the plurality of pores has an average pore size in the range of about 40 nm to about 100 nm. Some embodiments provide porous materials containing pores with an average pore size of about 10 nm or greater may include, within the pores, a pharmaceutically active species in crystal form.

Some specific examples of porous materials are shown in Table 1.

TABLE 1
Examples of porous materials.

| Material | Pore size (nm) | Form |
|---|---|---|
| microcrystalline cellulose | 5-1000 | powder/granules |
| Cellulose/cellulose acetate | 200-1000 | Membranes |
| porous polymer matrix (such | 10-100 | Beads |
| as styrene or methacrylic acid | | |
| or divinyl benzene or a | | |
| combination thereof) | | |
| Mesoporous silicas | 2-15 | Powder |
| Mesoporous Carbon | 10 | Powder |
| Silicon Dioxide/Titanium | 2-50 | granules/powder |
| Dioxide/Aluminum oxide | | |
| controlled pore glass | 0.1-300 | Powder |
| Anodic aluminum oxide | 20-200 | membranes (60um |
| | | thick) |

The pharmaceutical compositions, formulations, and other materials described herein may optionally include other components suitable for use in a particular application. Examples of such components include, but are not limited to, binders, disintegrants, fillers, lubricants, solvents, surfactants, diluents, salts, buffers, emulsifiers, chelating agents, antioxidants, and the like.

Having thus described several aspects of some embodiments of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

### EXAMPLES

General Procedure: First, active pharmaceutical ingredients (APIs) were impregnated within the nanosized pores of the material of interest. Then, these molecules are induced to form a solid in the nanosized pores, resulting in the generation of nanosized crystalline or amorphous APIs confined within the pores of the excipient (or other biologically compatible) material. One such example of how this may be achieved is as follows: the API is typically dissolved into an appropriate solvent generating a solution and the solution is then placed in contact with the porous material. The solution is allowed to impregnate the pores of the excipient material by an equilibration/diffusion process or is otherwise engineered to fill the pores. The solution is removed from the surface of the particles by washing. The solution remaining in the pores is then brought into conditions of supersaturation (e.g. cooling, anti-solvent addition or evaporation) in order to induce precipitation/crystallization of the API confined within the pores of the material. This has been exemplified with the API ibuprofen and selected nanoporous materials. Such methods may be used individually or in combination with either a batch or continuous processing manner.

### Example 1

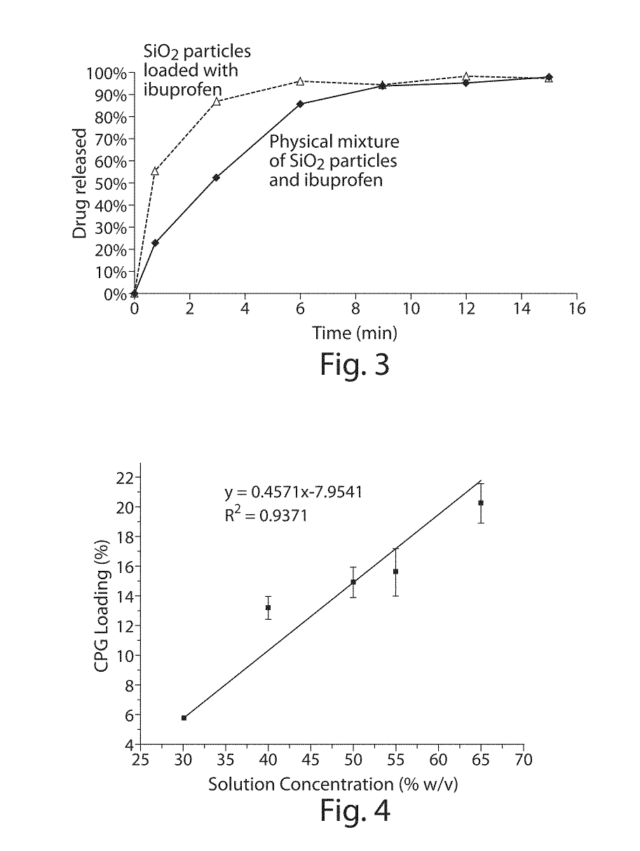

The following example describes the formation of nano-crystalline APIs in porous silicon dioxide particles. An under saturated API solution was prepared by combining 5 g ibuprofen with 10 mL ethanol. Porous silicon dioxide particles (1 g, pore size of about 40 nm) were placed in a 50 ml Buchner flask, which was sealed with a rubber cap and connected to a vacuum line. The flask was placed under reduced pressure (about 0.5 atm) in order to reduce the trapping of air inside the pores during the API-loading process. The API solution was injected into the flask through the rubber cap using a syringe and needle. To enhance mass transfer, the flask was lightly shaken and then kept still for 60 minutes. Afterwards, the API-loaded silicon dioxide particles were filtered and washed. After two-weeks of slow evaporation, the API within the pores was characterized by X-ray powder diffraction (XRPD) and differential scanning calorimetry (DSC). The results indicated the existence of crystalline forms. The API loading reached up to about 22 wt. %, based on the weight of the silicon dioxide particles. FIG. 3 shows a graph of the dissolution tests of nano-crystalline ibuprofen loaded inside porous silicon dioxide particles (pore size of about 40 nm) compared to a physical mixture of crystalline ibuprofen and porous silicon dioxide particles.

### Example 2

The following example describes the formation of nano-crystalline fenofibrate in porous silicon dioxide particles. The same procedure as in Example 1 was employed. The API inside the pores was characterized by XRPD and DSC, showing the existence of crystalline forms. The API loading reached up to about 23 wt. %, based on the weight of the silicon dioxide particles.

### Example 3

The following example describes the formation of nano-crystalline griseofulvin in porous silicon dioxide particles. The same procedure as in Example 1 was employed. The API inside the pores was characterized by XRPD and DSC, showing the existence of crystalline forms. The API loading reached up to about 32 wt. %, based on the weight of the silicon dioxide particles.

### Example 4

The following example describes the formation of amorphous APIs in porous silicon dioxide particles. The same procedure as in Example 1 was employed; however, rather than controlling the rate of evaporation, the particles were exposed to ambient air overnight for crystallization. The API inside the pores was characterized by XRPD and DSC, showing no evidence of crystalline materials. The API loading reached up to about 20 wt. %, based on the weight of the silicon dioxide particles.

### Example 5

The following example describes the formation of amorphous indomethacin in porous silicon dioxide particles. The same procedure as in Example 1 was employed. The API inside the pores was characterized by XRPD and DSC, showing no evidence of crystalline forms. The API loading reached up to about 18 wt. %, based on the weight of the silicon dioxide particles.

### Example 6

The following example describes the formation of nano-crystalline APIs in porous cellulose membranes by spraying. An under saturated API solution was prepared by combining 5 g ibuprofen with 10 ml ethanol. Solution droplets (microsized diameter) of the API solution were sprayed by Buchi Nano-spray-dryer (Model: B90) and dispensed onto the surface of a cellulose membrane (200 nm pore size). Given the hydrophilicity of the cellulose membrane, the solution droplets diffused into the pores for crystallization. The API inside the pores was characterized by XRPD and DSC and determined to be a crystalline material. The API loading reached up to about 27 wt. %, based on the weight of the cellulose membrane.

### Example 7

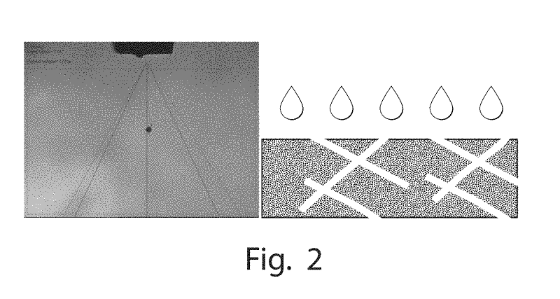

The following example describes the formation of nano-crystalline APIs in porous cellulose membranes by nano-plotting. An under saturated API solution was prepared by combining 5 g ibuprofen with 10 ml ethanol. Solution droplets of 0.1-1 nL were generated by GeSiM Nano-Plotter® (Model: NP2.1) and dispensed onto the surface of a cellulose membrane (200 nm pore size). (FIG. 2) Given the hydrophilicity of the cellulose membrane, the solution droplets diffused into the pores for crystallization. The API inside the pores was characterized by XRPD and DSC and determined to be a crystalline material. The API loading reached up to about 15 wt. %, based on the weight of the cellulose membrane.

### Example 8

The following is an example of a process for production. A vessel was filled with 1 g of biocompatible controlled pore glass (CPG). The vessel was subject to vacuum, evacuated and then an under saturated solution containing ibuprofen and ethanol (30% w/v) was pumped into the vessel to allow the solution to fill the pores of CPG. After waiting for a set amount of time the solution was drained from the vessel. A cold rinse of ˜10 ml of ethanol solvent was applied to the material in the vessel and was quickly drawn off under vacuum. Air was then flowed and distributed throughout the vessel, increasing flow rate over time, in order to dry and crystallize ibuprofen within the CPG material. X-ray diffraction (XRD) and DSC confirmed the preparation of nano-crystals of ibuprofen within the CPG and thermo-gravimetric analysis (TGA) was used to measure the amount of ibuprofen loaded. The amount of ibuprofen in the CPG was 6 wt. %. This process was repeated with under saturated solutions of different concentration and showed a linear increase in the loading with concentration in the range tested. (FIG. 4)

### Example 9

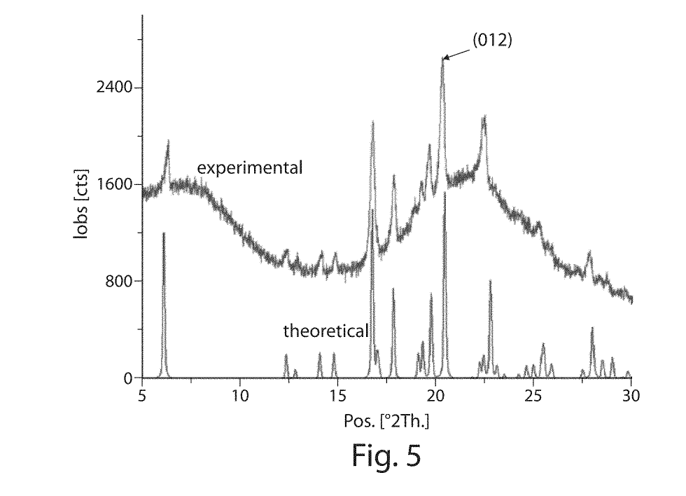

The following example describes X-ray powder diffraction (XRPD) analysis of porous CPG containing crystalline ibuprofen within the pores (as produced in Example 8). FIG. 5 shows the X-ray powder diffraction (XRPD) pattern of CPG containing crystalline ibuprofen (IBP) compared to that of the theoretical pattern for form I IBP (CCDC refcode. IBPRACO2). As shown in FIG. 5, the material includes an amorphous porous phase of SiO2 with mean pore diameter of 110 nm, and crystalline form I ibuprofen is shown to have crystallized within these pores. The Scherrer equation was used to estimate the particle size of IBP crystals within the pores from the peak broadening associated with the (012) peak of form I IBP2 measured at 20.5 °2θ. This resulted in an estimated average particle size of 66 nm, which is less than the pore size of the CPG, suggesting that IBP nanocrystals are confined within the pores.

### Example 10

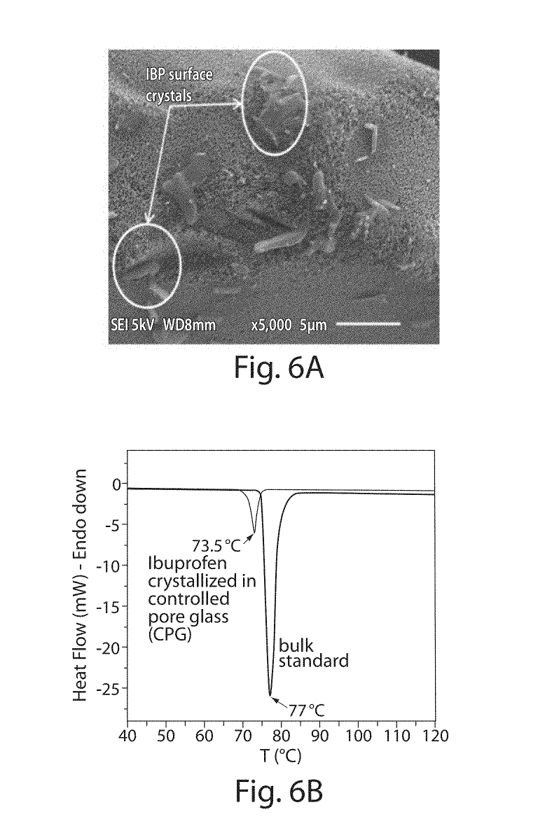

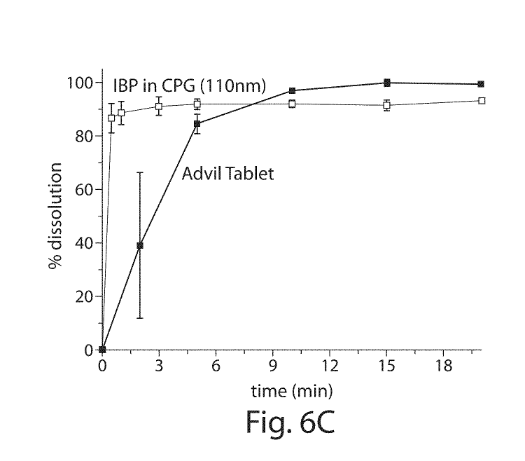

The following example describes the study of a CPG particle after impregnation with crystalline IBP using scanning electron microscopy (SEM) and differential scanning calorimetry (DSC). (FIG. 6) As shown by the SEM image in FIG. 6A, bulk-sized crystals (>2 microns) of IBP were observed on the exterior surface of the CPG, but the exterior surface was otherwise substantially free of IBP bulk crystals.

As size-dependent melting point depression is a characteristic of nanosized crystals, DSC was used to measure the melting point (Tₘ) for bulk IBP and for IBP that was crystallized in CPG. The thermogram in FIG. 6B shows a Tₘ event for bulk IBP occurring at 77° C. whereas a single Tₘ for IBP crystallized in CPG was recorded to be 73.5° C., giving a ΔTₘ˜4.5° C. Such a shift in melting point is typically expected for crystals in the nanosize range (e.g., <100 nm). Furthermore, a single melting point event for IBP crystallized in CPG occurring below the Tₘ of the bulk sized IBP crystals indicated that the sample contained a vast majority of nanosized crystals only, i.e., that the sample was substantially free of bulk-sized crystals.

FIG. 6C shows a comparison between the dissolution rate of IBP nanocrystals in CPG having a mean pore diameter of 110 nm (loading ˜200mg) and the marketed 200 mg IBP formulated tablet known as Advil®. The dissolution rates of each were measured using a USP II apparatus with aqueous dissolution media (phosphate buffer at pH 7.2).

### Example 11

The following example demonstrates the increase in dissolution rate of pharmaceutically active species when arranged within pores of a porous material, relative to bulk-sized crystals of the same pharmaceutically active species or formulations of the same pharmaceutically active species.

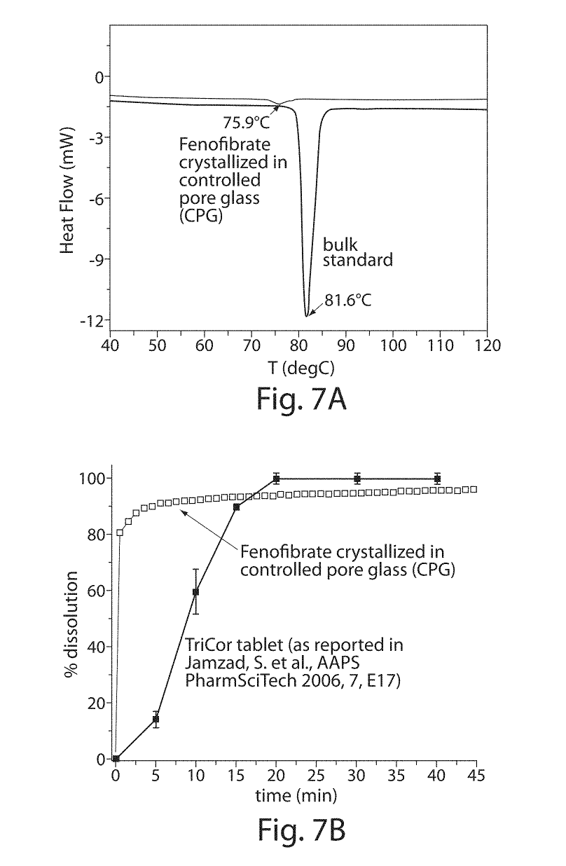

CPG with a mean pore diameter of 110 nm was prepared to contain nanocrystalline fenofibrate (FEN), which exhibits poor solubility in water, according to the method described in Example 8. FIG. 7A shows a DSC thermogram comparing the melting points of FEN crystallized within the CPG and bulk-sized crystals (>2 μm) of FEN. The Tₘ event for FEN crystallized in CPG occurred at a significantly lower temperature than the bulk-sized FEB standard, giving a melting point depression, ΔTₘ, ˜6° C. The dissolution rate of the FEN crystallized in CPG was measured using a USP II apparatus with aqueous dissolution media (containing 0.72% w/v sodium dodecylsulphate at pH 6.8). As shown in FIG. 7B, an extremely fast dissolution rate was observed for the FEN crystallized in CPG, with 90% dissolution of the FEN in ˜3 min.

As a comparison, TriCor tablets of FEN were formed using nanomilling technology that reduces the particle size of FEN to ˜400 nm, according to the methods described in Jamzad, S. et al., AAPS PharmSciTech 2006, 7, E17. The dissolution rate of the TriCor FEN tablets was also measured using the methods and conditions described in Jamzad, S. et al., AAPS PharmSciTech 2006, 7, E17. 90% Dissolution of the tablets was observed in ˜15 min, which was significantly slower than the dissolution rate observed for FEN crystallized in CPG. This demonstrates that a significant increase in dissolution rate for nanosized crystals of pharmaceutically active species contained with nanoporous material.

### Example 12

This example describes the crystallization of APIs in rigid nanoporous media over a broad range of pore sizes. The API fenofibrate, which is known in two polymorphic forms, was crystallized over a range of pore sizes (10 different pore sizes between 12 nm-300 nm) of CPG and a biocompatible fumed silica AEROPERL®. The drug loadings were determined with thermogravimetric analysis (TGA) and the nanocrystal melting points and enthalpies of fusion were studied with differential scanning calorimetry (DSC). Crystallinity was assessed with X-ray powder diffraction (XRPD), while both polymorphism and degree of crystallinity was studied using solid-state nuclear magnetic resonance (ssNMR).

Materials: Fenofibrate (FEN) was obtained from Xian Shunyi Bio-chemical Technology Company. Silicon dioxide (silica) particles of varying pore sizes were obtained from three sources. AEROPERL®, a colloidal fumed silica, was obtained from Evonik USA, according to whom the material fulfils requirements of the European Pharmacopeia as well as the United States Pharmacopeia and the National Formulary. AEROPERL® consists of bead-like mesoporous granules with a pore size of ˜35 nm. Controlled pore glass (CPG) was obtained from Millipore in pore sizes of 300 nm and 70 nm. CPG was also obtained from Prime Synthesis in pore sizes of 191.4 nm, 151.5 nm, 105.5 nm, 53.7 nm, 38.3 nm, 30.7 nm, 20.2 nm, and 12.7 nm.

Experimental Apparatus: (1) A small amount (˜0.25 g) of CPG (or AEROPERL®) was placed in a 20 mL scintillation vial, resulting in a CPG bed height of about 0.3 cm and a top surface area of ˜3.1 cm2. In this example, the preparation of 0.25 g of CPG to be loaded with drug was plenty for analytical purposes. (2) The pore volume present in the entire CPG sample was then calculated based on the given pore volume/gram CPG. A 60% weight/volume solution of fenofibrate in ethyl acetate was prepared. API solution in equal amount to the pore volume present in the CPG was then micropipetted over the surface of the CPG in the scintillation vial as uniformly as possible. (3) Immediately after pipetting, a metal spatula was used to stir the mixture, to wet as much of the CPG as possible, ceasing only when the mixture appeared dry. The drug-loaded CPG was then left in a fume hood for an additional 24 hrs to continue evaporation of excess solvent. It is noteworthy that no wash step was required in this method. Samples were prepared in triplicate for each pore size.

X-Ray Powder Diffraction Analysis: X-Ray powder diffraction (XRPD) was performed on all samples using a PANalytical X'Pert PRO diffractometer at 45 kV with an anode current of 40 mA. The instrument has a PW3050/60 standard resolution goniometer and a PW3373/10 Cu LFF DK241245 X-ray tube. Samples were placed on a spinner stage in reflection mode. Settings on the incident beam path included: soller slit 0.04 rad, mask fixed 10 mm, programmable divergence slit and fixed ½° anti-scatter slit. Settings on the diffracted beam path include: soller slit 0.04 rad and programmable anti-scatter slit. The scan was set as a continuous scan: 2θ angle between 4 and 40°, step size 0.0167113° and a time per step of 31.115 s.

Differential Scanning Calorimetry Analysis: A Q2000 instrument from TA instruments was utilized for the differential scanning calorimetry (DSC) analysis. Inert atmosphere environment was maintained in the sample chamber using a nitrogen gas cylinder set to a flow rate of 50 ml/min. An extra refrigerated cooling system (RCS 40, TA instruments) was used to broaden the available temperature range between −40 and 400° C. Tzero® pans and lids were used with ˜5 mg of sample. A heating rate of 10° C./min was applied and the samples were scanned from −20 to 180° C. When determining the enthalpy of fusion for a given sample, the DSC curve was integrated for 30° C. centred on the melting temperature of each pore size to capture the entire melting event.

Thermogravimetric Analysis: Thermogravimetric analysis (TGA) was performed on a Q500 instrument from TA instruments connected with a nitrogen gas cylinder to maintain a flow rate of 25 mL/min to keep the sample chamber under an inert gas environment. Between 5 and 10 mg of sample were loaded on platinum sample pans from TA instruments. The samples were allowed to equilibrate at 30° C. and then heated at 10° C./min to 300° C.

Solid-state Nuclear Magnetic Resonance: Solid-state nuclear magnetic resonance experiments were conducted on a homebuilt 500 MHz spectrometer. Prepared samples were packed into Revolution NMR (Fort Collins, USA) 4 mm o.d. (60 ul fill volume) ZrO2 rotors, equipped with Vespel drive and top caps. Spectra were acquired on a 4 mm Chemagnetics triple resonance (1H/13C/15N) magic-angle spinning (MAS) probe. ¹³C natural abundant spectra were acquired using cross-polarization (CP), a recycle delay of 3 seconds, between 16,384 and 65,536 co-added transients and a spinning frequency of 9,000±3 Hz. The Hartman-Hahn match condition was optimized by setting 1H to 50 kHz (γB½π), a positive ramp contact pulse for ¹³C (centered at 58 kHz) and a contact time of 1.5 ms. All data were acquired using TPPM 1H decoupling (100 kHz, 1H γB½π). The magic-angle was adjusted using potassium bromide (KBr) at a spinning frequency of 5 kHz, (rotational echoes >11.5 ms). ¹³C spectra were referenced (and shimmed, FWHM=4 Hz) using solid adamantane to 40.49 ppm (high frequency resonance) with respect to DSS (0 ppm).

Dissolution test: The dissolution tests were designed following USP standards. Analysis of the percentage of dissolved API was done using built-in ultraviolet-visible spectroscopy at 286 nm. The dissolution buffer used was 0.025 M sodium dodecyl sulfate solution (7.21 grams of powdered SDS (Sigma Aldrich) was dissolved and brought up to 1000 mL in water). The dissolution profile of the sample was determined using USP Dissolution Apparatus 2 at 37° C. The apparatus operated at 75 RPM. 900 mL of the buffer solution was allowed to reach the equilibrium temperature before sample was placed in the apparatus. Enough sample of API-loaded CPG was added such that the targeted concentration of fenofibrate in solution was 15 μg/mL, within the expected linear range. Samples of both uncrushed and crushed bulk fenofibrate were analyzed as comparison. Samples were acquired for about 29 hours.

Results: Fenofibrate was selected as a model API to work within preliminary studies. It is poorly water soluble, <1 mg/mL at 37° C. [30] and has two known polymorphs, crystalline form I with a melting point around 80° C. and a metastable form II with a melting point around 73° C. The metastable form has been collected in a sample of amorphous fenofibrate that was heated to around 40° C. Fenofibrate was chosen for initial studies due to its lack of multiple stable polymorphs; it is advantageous to first study how a single polymorph changes with varying crystal size. Table 1 summarizes the sizes of CPG and AEROPERL® used and the pore volumes as provided by the supplier.

TABLE 1
Pore sizes and volumes of porous silica as provided by the producer

| Pore Size | Pore Volume | |
|---|---|---|
| (nm) | (cc/gram) | Producer |
| 300 | >1 | Millipore |
| 191.4 | 1.5 | Prime Synthesis |
| 151.5 | 1.2 | Prime Synthesis |
| 105.5 | 1.4 | Prime Synthesis |
| 70 | >1 | Millipore |
| 53.7 | 1.3 | Prime Synthesis |
| 38.3 | 1.3 | Prime Synthesis |
| 30.7 | 1.11 | Prime Synthesis |
| 20.2 | 1.12 | Prime Synthesis |
| 12.7 | 0.5 | Prime Synthesis |
| Aeroperl | 1.6 | Evonik |
| (~35) | | |

TABLE 2
Crystalline fenofibrate loaded in porous silica particles

| Pore | FEN mass | Melting point | Polymorph | Polymorph |
|---|---|---|---|---|
| size (nm) | loaded (wt. %) | by DSC (° C.) | by XRPD | by ssNMR |
| 300 | 29.4 ± 1.2 | 79.9 ± 0.1 | Form I | Form I |
| 191.4 | 40.0 ± 2.0 | 79.8 ± 0.5 | Form I | Form I |
| 151.5 | 31.5 ± 1.0 | 79.0 ± 0.2 | Form I | Form I |
| 105.5 | 35.7 ± 0.5 | 78.7 ± 0.2 | Form I | Form I |
| 70 | 28.1 ± 0.4 | 77.7 ± 0.2 | Form I | Form I |
| 53.7 | 33.4 ± 0.4 | 75.2 ± 0.6 | Form I | Form I |
| 38.3 | 29.4 ± 0.4 | 71.8 ± 0.5 | Form I | Form I |
| ~35 | 28.0 ± 1.7 | 70.3 ± 0.8 | Form I | Form I |
| 30.7 | 29.4 ± 0.7 | 71.2 ± 0.1 | Form I | Form I |
| 20.2 | 26.2 ± 0.8 | 64.2 ± 0.4 | Form I | Form I |

All loading data, melting points, and polymorph observations via XRPD and ssNMR are summarized in Table 2. High drug loadings were achieved via the method of applying the pore volume of drug solution. In the XRPD samples, there was a large amorphous feature which disrupted the baseline (to be subtracted) due to the amorphous silica matrix which made up the bulk of the sample. NMR was isotope selective and invariant to the substrate that the API was placed upon offering an approach to probe the degree of crystallinity and identify polymorphs easily using ¹³C CP MAS NMR.

Fenofibrate in 20 to 300 nm CPG illustrated clean ¹³C spectra with high crystalline API formation. DSC and XRPD data indicated an inability to crystallize fenofibrate in the 12 nm CPG, suggesting an amorphous form (vide infra). In examining the literature, it has been reported that the pore diameter should be at least 20 times the molecular diameter for crystallization in confined spaces. Fenofibrate has an estimated molecular size of 0.98-1.27 nm. It was hypothesized that this was the reason why the 12 nm CPG showed no crystalline fenofibrate in the powder x-ray diffraction results, as it is less than 20 times the diameter of fenofibrate. It was postulated that under slow crystallization conditions, crystals could be formed in pore sizes under 20 times the molecular diameter, which would explain the combination of broadened (i.e., amorphous phase) and narrow (i.e., crystalline) ¹³C resonance observed in the 12 nm sample.

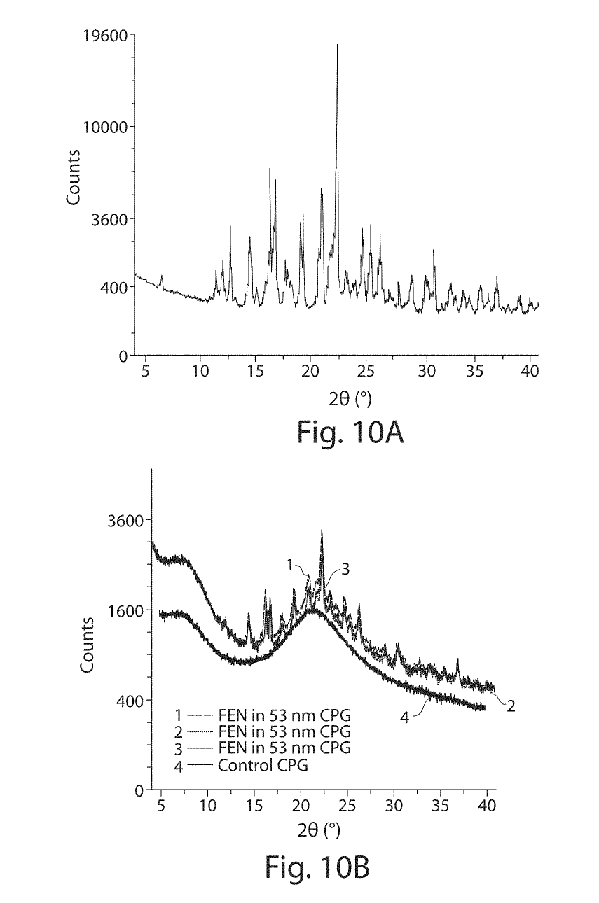

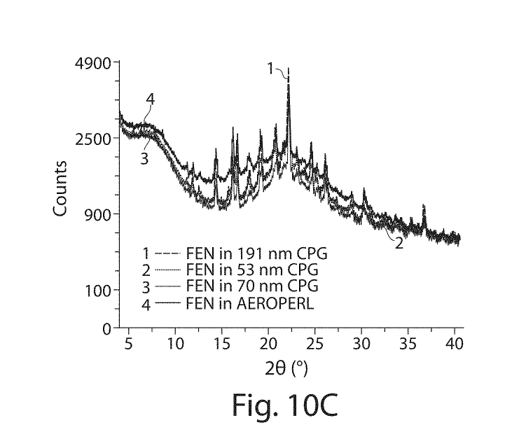

Crystal form identification with XRPD: With the exception of fenofibrate in 12 nm CPG which showed no crystallinity, all samples showed the same XRPD peak pattern, both within trials of the same size CPG and across different sizes of CPG. FIG. 10A is a scan of bulk fenofibrate and FIG. 10B shows the XRPD scans of a single representative size of 53 nm CPG, across all three trials. It was evident that the crystal pattern was consistent throughout trials of a given pore size, which was also seen in all other pore sizes. FIG. 10C shows an overlay of scans from three representative CPG sizes (191, 53, and 70 nm) and AEROPERL® which show the same pattern across pore sizes. Note that in the overlay of all CPG sizes, AEROPERL® which has a different background signal than the CPG, which was to be expected. Crystalline fenofibrate form I has reported theoretical diffractogram main peaks at 12° (2θ), 14.5° (2θ), 16.2° (2θ), 16.8° (2θ), and 22.4° (2θ). The identity of all samples of nanocrystalline fenofibrate as form I could be confirmed by matching peaks and the absence of other peak positions. ¹³C Cross-polarization MAS NMR spectra for all fenofibrate loaded porous silica particles were used to identify amorphous or crystalline fenofibrate and identify whether the crystalline phase present were form I or II. All ¹³C MAS NMR spectra illustrate highly crystalline fenofibrate (form I), with line widths between 60 and 85 Hz. Isotropic chemical shift data for silica particles with pore sizes ranging between 20 and 300 nm revealed identical spectra with no evidence of structural disorder. The slight decrease in resolution (¹³C line broadening from 300 to 20 nm) is due to the increase of surface disorder as the nanocrystals become increasingly smaller (i.e., surface vs nanocrystalline core).

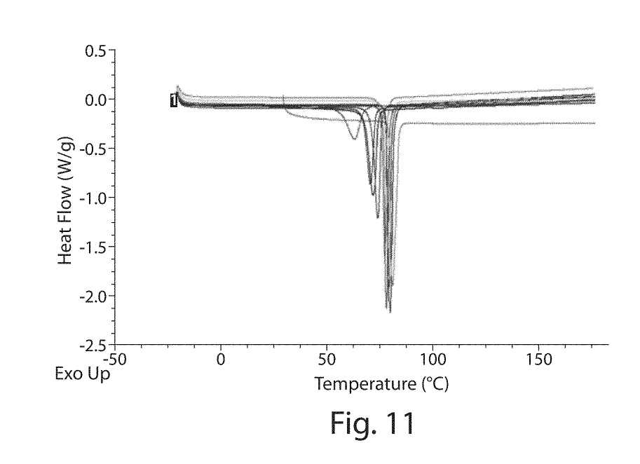

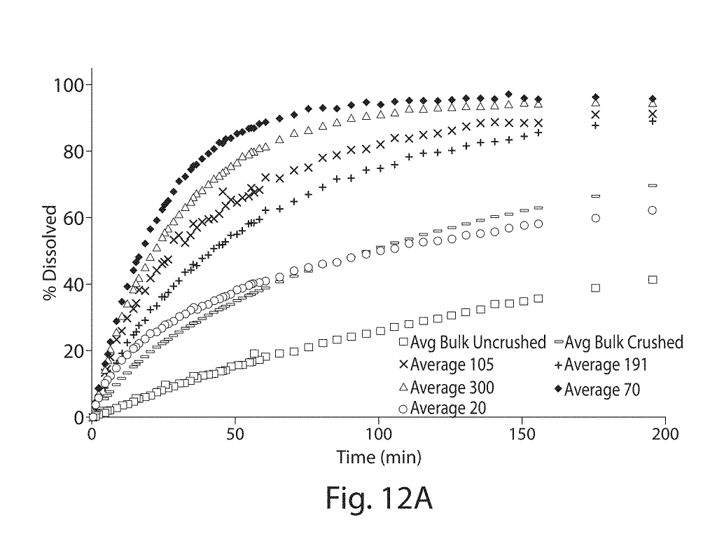

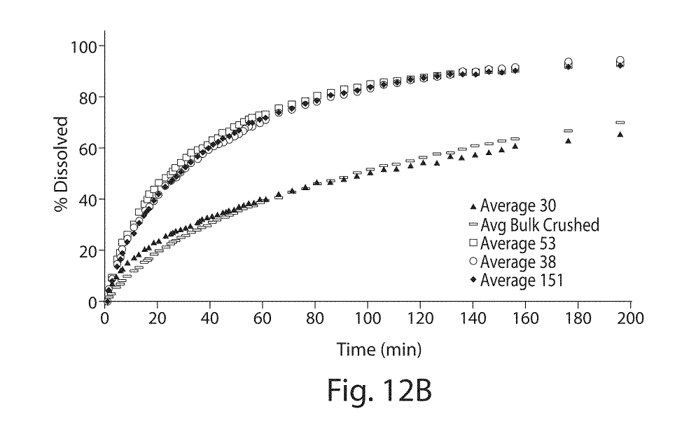

The melting point of bulk fenofibrate crystals was measured and found to be 81.6±0.2° C. FIG. 11 shows an overlay of the DSC scans for representative trials of fenofibrate crystallized in each CPG pore size. Individual, sharp peaks can be found at decreasing melting point temperatures, moving left as the CPG pore size decreases. Double peaks were not seen in the trials, indicating the method was successful inhibiting the formation of any surface crystals.

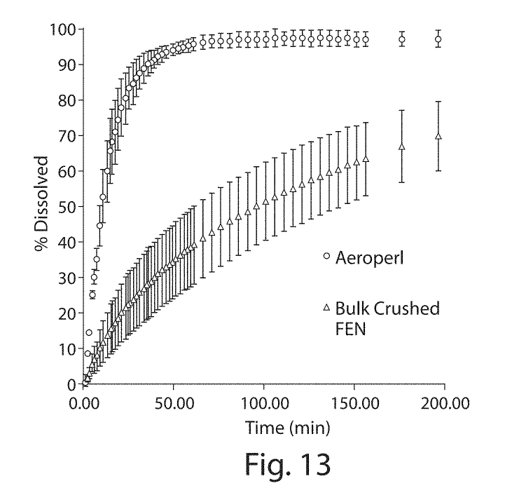

Dissolution profiles were tested and shown in FIGS. 15A-15B. The nanocrystalline fenofibrate with the most enhanced dissolution profile occurred in the AEROPERL® matrix, shown in FIG. 13. AEROPERL® showed a roughly 10 fold increase in dissolution rate compared with crushed bulk fenofibrate. It reached >80% dissolution in 22.5 minutes where crushed bulk fenofibrate reached >80% dissolution in 295.5 minutes. Fenofibrate nanocrystals confined to 20 and 30 nm CPG had profiles which aligned closely with the crushed bulk profile indicating that, at small pore sizes, diffusional resistance likely matters to enhancing dissolution rate. Nanocrystals in CPG above 30 nm showed improved dissolution over the bulk crushed and uncrushed fenofibrate crystals at all time points of the study. The dissolution profiles can be clustered into two groups based on manufacturer. The 70 nm and 300 nm (Millipore CPG) confined fenofibrate nanocrystals were the next most enhanced profiles after AEROPERL® and showed the expected faster dissolution with smaller pore/crystal size. The fenofibrate nanocrystals confined to the other pore sizes (Prime Synthesis CPG) all had very similar, still improved, dissolution profiles with no discernible trend by pore size. It is likely that the differences in pore geometry and tortuosity of AEROPERL® and the two types of CPG contribute to the differences in improvement in dissolution rate seen in the study.

### Example 13

This example describes a continuous two stage process for the loading of controlled pore glass (CPG) with fenofibrate, formation of crystalline solid fenofibrate in the pores of the controlled pore glass such that the exterior surface of the controlled pore glass was substantially free of crystals, and growth of the crystals after formation to increase the loading of API. Controlled pore glass with pores sizes of 191.4 nm, 151.5 nm, 105.5 nm, 53.7 nm, and 38.3 nm were used.

The process shown in FIG. 9 was used to form and grow fenofibrate crystals. The first stage consisted of loading of fenofibrate into the pores and crystallization within the pores of the controlled pore glass. Briefly, controlled pore glass and a 60% weight/volume solution of fenofibrate in ethyl acetate were feed into a mixed suspension mixed product removal (MSMPR) device and the resulting suspension was mixed in the MSMPR device to allow for loading. After a period of time sufficient for impregnation of fenofibrate within CPG pores, the impregnated CPG was removed from the MSMPR device via filtration, washed to remove fenofibrate on the surface of the CPG, and the fenofibrate within CPG pores was crystallized. The second stage consisted of growing the crystals within the CPG pores formed in the first stage. Briefly, the controlled pore glass having crystalline fenofibrate within the pores and a supersaturated solution of fenofibrate were feed into a second mixed suspension mixed product removal device. The second MSMPR device was maintained under conditions suitable for crystal growth and not spontaneous nucleation. The supersaturated solution of fenofibrate utilized did not have a concentration within the metastable zone necessary for spontaneous nucleation of fenofibrate. Accordingly, crystal growth within the pores occurred without the formation of crystals on the exterior surface of the CPG. After crystal growth, the controlled pore glass having crystalline fenofibrate within the pores was removed from the second MSMPR device, filtered, washed, and dried. A one stage process consisting of only the first stage described above was performed as a control.

The two stage process lead to a higher API weight percentage and relative percent loading compared to the one stage process. The theoretical maximum weight percentage and the actual weight percentages for the one stage and two stage processes are shown in Table 3. The relative percent loading of the two stage process was greater than about 80% while the loading efficiency of the one stage process was about 50% to about 70%.

TABLE 3
Crystalline fenofibrate loaded in controlled pore glass (CPG) particles

| | Pore | | Actual weight | Actual weight |
|---|---|---|---|---|
| CPG | volume | Theoretical weight | percent | percent |
| (nm) | (cc/g) | percent | (One Stage) | (Two Stages) |
| 300 | 1.0 | 54% | 33.2% | — |
| 191 | 1.5 | 63.9% | 36% | 54.87% |
| 151 | 1.2 | 58.6% | 37.6% | 52.51% |
| 105 | 1.4 | 62.3% | 34.7% | 55.33% |
| 53 | 1.3 | 60.5% | 40.1% | 51.38% |
| 38 | 1.3 | 60.5% | 41.5% | 52.27% |

What is claimed:

1. A method for forming a material comprising a pharmaceutically active species, comprising:
contacting a porous material comprising a plurality of pores, with a pharmaceutically active species, such that the pharmaceutically active species enters the pores;
placing the porous material under a set of conditions which facilitates formation of a crystal of the pharmaceutically active species; and
allowing the pharmaceutically active species to form a crystal within the plurality of pores,
wherein, upon formation of the crystals within the plurality of the pores, the exterior surface of the porous material is substantially free of crystals of the pharmaceutically active species having a size of 1 micron or greater.

2. A method as in claim 1, further comprising filtering and/or washing the porous material before formation of the crystal.

3. A method as in claim 1, further comprising filtering and/or washing the porous material after formation of the crystal.

4. A method as in any preceding claim, wherein the step of contacting comprises combining a solution comprising the pharmaceutically active species and a fluid carrier with the porous material.

5. A method as in claim 4, wherein the solution further comprises a surfactant.

6. A method as in claim 4, wherein the solution is in the form of droplets.

7. A method as in claim 1, wherein the step of contacting comprises exposure to ambient pressure.

8. A method as in claim 1, wherein the step of contacting comprises placing the porous material and pharmaceutically acceptable carrier under reduced pressure.

9. A method as in claim 1, wherein the step of contacting comprises heating the porous material and pharmaceutically acceptable carrier.

10. A method as in claim 1, wherein the step of contacting comprises cooling the porous material and pharmaceutically acceptable carrier.

11. A method as in claim 1, wherein the step of contacting comprises sonicating the porous material and pharmaceutically acceptable carrier.

12. A method as in claim 1, wherein the set of conditions comprises removing at least a portion of the fluid carrier.

13. A method as in claim 1, wherein the set of conditions comprises removing substantially all of the fluid carrier.

14. A method as in claim 1, wherein the set of conditions comprises adding a fluid carrier that facilitates formation of a crystal of the pharmaceutically active species.

15. A method as in any preceding claim, wherein the porous material is a biologically compatible porous material.

16. A method as in any preceding claim, wherein the porous material comprises cellulose, cellulose acetate, carbon, silicon dioxide, titanium dioxide, aluminum oxide, or another glass material.

17. A method as in any preceding claim, wherein the plurality of pores has an average pore size of about 10 nm or greater.

18. A method as in any preceding claim, wherein the plurality of pores has an average pore size in the range of about 10 nm to about 1000 nm, about 10 nm to about 500 nm, about 10 nm to about 250 nm, about 10 nm to about 100 nm, or about 30 nm to about 100 nm.

19. A method as in claim 18, wherein the plurality of pores has an average pore size in the range of about 30 nm to about 100 nm.

20. A method as in any preceding claim, wherein, in the absence of association with the porous material, the pharmaceutically active species is substantially insoluble in aqueous solutions.

21. A method as in any preceding claim, wherein, in the absence of association with the porous material, the pharmaceutically active species when having a particle size greater than about 1000 nm has a solubility of less than 0.1 mg/mL in aqueous solution at room temperature.

22. A method as in any preceding claim, wherein the pharmaceutically active species is ibuprofen, deferasirox, felodipine, griseofulvin, bicalutamide, glibenclamide, indomethacin, fenofibrate, itraconazole, or ezetimibe.

23. A method as in any preceding claim, comprising:
combining a solution comprising the pharmaceutically active species and a fluid carrier with the porous material under ambient conditions such that the pharmaceutically active species enters the pores;
filtering and/or washing the porous material containing the pharmaceutically active species within the pores;
placing the porous material under the set of conditions which facilitates formation of a crystal of the pharmaceutically active species; and
allowing the pharmaceutically active species to form the crystal within the plurality of pores.

24. A method as in any preceding claim, comprising:
combining a solution comprising the pharmaceutically active species and a fluid carrier with the porous material at a pressure greater than 1 atm such that the pharmaceutically active species enters the pores;
filtering and/or washing the porous material containing the pharmaceutically active species within the pores;
placing the porous material under the set of conditions which facilitates formation of a crystal of the pharmaceutically active species; and
allowing the pharmaceutically active species to form the crystal within the plurality of pores.

25. A method as in any preceding claim, comprising:
combining a solution comprising the pharmaceutically active species and a fluid carrier with the porous material under reduced pressure such that the pharmaceutically active species enters the pores;
filtering and/or washing the porous material containing the pharmaceutically active species within the pores;
placing the porous material under the set of conditions which facilitates formation of a crystal of the pharmaceutically active species; and
allowing the pharmaceutically active species to form the crystal within the plurality of pores.

26. A method as in any preceding claim, comprising:
sonicating a solution comprising the pharmaceutically active species and a fluid carrier and the porous material such that the pharmaceutically active species enters the pores;
filtering and/or washing the porous material containing the pharmaceutically active species within the pores;
placing the porous material under the set of conditions which facilitates formation of a crystal of the pharmaceutically active species; and
allowing the pharmaceutically active species to form the crystal within the plurality of pores.

27. A method as in any of claims 23-26, wherein the solution further comprises a surfactant.

28. A method as in any one of claims 23-27, wherein the solution is in the form of droplets.

29. A method as in any preceding claim, comprising:
combining the pharmaceutically active species in solid form with the porous material at a temperature at or above the melting temperature of the pharmaceutically active species and below the melting temperature of the porous material, such that the pharmaceutically active species enters the pores;
cooling the porous material and pharmaceutically active species to facilitate formation of a crystal of the pharmaceutically active species; and
filtering and/or washing the porous material containing the pharmaceutically active species within the pores.

30. A method comprising any preceding claim, further comprising applying centrifugal force to the porous material and pharmaceutically active species in order to remove oxygen, if present, within the pores.

31. A method comprising any preceding claim, further comprising the step of compressing the porous material containing the pharmaceutically active species in crystal form into a tablet.

32. A method comprising any preceding claim, further comprising the step of placing the porous material containing the pharmaceutically active species in crystal form within a capsule.

33. A method comprising any preceding claim, wherein 80% dissolution of the pharmaceutically active species in crystal form within the pores occurs at least about 10% faster than that of the pharmaceutically active species in crystal form that is not within the pores and that has a particle size greater than about 1000 nm.

34. A method comprising any preceding claim, wherein 80% dissolution of the pharmaceutically active species in crystal form within the pores occurs at least about 20% faster than that of the pharmaceutically active species in crystal form that is not within the pores and that has a particle size greater than about 1000 nm.

35. A method as in any preceding claim, wherein the method is carried out as a batch, semi-batch, or continuous process.

36. A material comprising a pharmaceutically active species, prepared by the method according to any preceding claim.

37. A material comprising a pharmaceutically active species, comprising:
a porous material comprising a plurality of pores having an average pore size of about 10 nm or greater; and
a pharmaceutically active species in crystal form positioned within the plurality of pores,
wherein the exterior surface of the porous material is substantially free of crystals of the pharmaceutically active species having a size of 1 micron or greater.

38. A material as in claim 37, wherein the porous material is a biologically compatible porous material.

39. A material as in claim 38, wherein the porous material comprises cellulose, cellulose acetate, carbon, silicon dioxide, titanium dioxide, aluminum oxide, or another glass material.

40. A material as in any one of claims 37-39, wherein the plurality of pores has an average pore size of about 10 nm or greater.

41. A material as in any one of claims 37-40, wherein the plurality of pores has an average pore size in the range of about 10 nm to about 1000 nm, about 10 nm to about 500 nm, about 10 nm to about 250 nm, about 10 nm to about 100 nm, or about 30 nm to about 100 nm.

42. A material as in claim 41, wherein the plurality of pores has an average pore size in the range of about 30 nm to about 100 nm.

43. A material as in any one of claims 37-42, wherein, in the absence of association with the porous material, the pharmaceutically active species is substantially insoluble in aqueous solutions.

44. A material as in any one of claims 37-43, wherein, in the absence of association with the porous material, the pharmaceutically active species when having a particle size greater than about 1000 nm has a solubility of less than 0.1 mg/mL in aqueous solution at room temperature.

45. A material as in any one of claims 37-44, wherein the pharmaceutically active species is ibuprofen, deferasirox, felodipine, griseofulvin, bicalutamide, glibenclamide, indomethacin, fenofibrate, itraconazole, or ezetimibe.

46. A material as in any one of claims 37-45, wherein 80% dissolution of the pharmaceutically active species in crystal form within the pores occurs at least about 10% faster than that of the pharmaceutically active species in crystal form that is not within the pores and that has a particle size greater than about 1000 nm.

47. A material as in any one of claims 37-46, wherein 80% dissolution of the pharmaceutically active species in crystal form within the pores occurs at least about 20% faster than that of the pharmaceutically active species in crystal form that is not within the pores and that has a particle size greater than about 1000 nm.

48. A pharmaceutical composition, comprising:
a porous material comprising a plurality of pores; and
a pharmaceutically active species in crystal form positioned within the plurality of pores; and
a pharmaceutically acceptable carrier,
wherein the exterior surface of the porous material is substantially free of crystals of the pharmaceutically active species having a size of 1 micron or greater.

49. A pharmaceutical composition as in claim 48, wherein the porous material is a biologically compatible porous material.

50. A pharmaceutical composition as in claim 49, wherein the porous material comprises cellulose, cellulose acetate, carbon, silicon dioxide, titanium dioxide, aluminum oxide, or another glass material.

51. A pharmaceutical composition as in any one of claims 48-50, wherein the plurality of pores has an average pore size of about 10 nm or greater.

52. A pharmaceutical composition as in any one of claims 48-51, wherein the plurality of pores has an average pore size in the range of about 10 nm to about 1000 nm, about 10 nm to about 500 nm, about 10 nm to about 250 nm, about 10 nm to about 100 nm, or about 30 nm to about 100 nm.

53. A pharmaceutical composition as in claim 52, wherein the plurality of pores has an average pore size in the range of about 30 nm to about 100 nm.

54. A pharmaceutical composition as in any one of claims 48-53, wherein, in the absence of association with the porous material, the pharmaceutically active species is substantially insoluble in aqueous solutions.

55. A pharmaceutical composition as in any one of claims 48-54, wherein, in the absence of association with the porous material, the pharmaceutically active species when having a particle size greater than about 1000 nm has a solubility of less than 0.1 mg/mL in aqueous solution at room temperature.

56. A pharmaceutical composition as in any one of claims 48-55, wherein the pharmaceutically active species is ibuprofen, deferasirox, felodipine, griseofulvin, bicalutamide, glibenclamide, indomethacin, fenofibrate, itraconazole, or ezetimibe.

57. A pharmaceutical composition as in any one of claims 48-56, wherein 80% dissolution of the pharmaceutically active species in crystal form within the pores occurs at least about 10% faster than that of the pharmaceutically active species in crystal form that is not within the pores and that has a particle size greater than about 1000 nm.

58. A pharmaceutical composition as in any one of claims 48-57, wherein 80% dissolution of the pharmaceutically active species in crystal form within the pores occurs at least about 20% faster than that of the pharmaceutically active species in crystal form that is not within the pores and that has a particle size greater than about 1000 nm.

59. A method as in any one of claims 1-36, further comprising placing the porous material under a second set of conditions, which facilitates growth of the crystal of the pharmaceutically active species, after formation of the crystal and growing the crystal of the pharmaceutically active species within the plurality of pores.

60. A method as in claim 59, wherein the second set of conditions does not facilitate spontaneous nucleation of the pharmaceutically active species.

61. A method as in claim 59 or 60, wherein after the growth step, the exterior surface of the porous material is substantially free of crystals of the pharmaceutically active species having a size of 1 micron or greater.

62. A method any one of claims 59-61, wherein the second set of conditions is different from the set of conditions.

63. A method as in any one of claims 59-62, wherein the relative percent loading of the pharmaceutically active species in the porous material after the growing step is greater than or equal to about 20%.

64. A method as in any one of claims 59-62, wherein the relative percent loading of the pharmaceutically active species in the porous material after the growing step is greater than or equal to about 50%.

65. A method as in any one of claims 59-62, wherein the relative percent loading of the pharmaceutically active species in the porous material after the growing step is greater than or equal to about 70%.

66. A method as in any one of claims 59-62, wherein the relative percent loading of the pharmaceutically active species in the porous material after the growing step is between about 30% and about 95%.

67. A method as in any one of claims 59-62, wherein the relative percent loading of the pharmaceutically active species in the porous material after the growing step is between about 70% and about 90%.

68. A method as in any one of claims 59-67, wherein the second set of conditions comprises combining a second solution comprising the pharmaceutically active species and a fluid carrier with the porous material.

69. A method as in any one of claims 59-68, further comprising filtering and/or washing the porous material after the growing step.

70. A material as in any one of claims 37-47, wherein the relative percent loading of the pharmaceutically active species in the porous material is greater than or equal to about 20%.

71. A material as in any one of claims 37-47, wherein the relative percent loading of the pharmaceutically active species in the porous material is greater than or equal to about 70%.

72. A material as in any one of claims 37-47, wherein the relative percent loading of the pharmaceutically active species in the porous material is between about 20% and about 90%.

73. A material as in any one of claims 37-47, wherein the relative percent loading of the pharmaceutically active species in the porous material is between about 70% and about 90%.

74. A pharmaceutical composition as in any one of claims 48-57, wherein the relative percent loading of the pharmaceutically active species in the porous material is greater than or equal to about 20%.

75. A pharmaceutical composition as in any one of claims 48-57, wherein the relative percent loading of the pharmaceutically active species in the porous material is greater than or equal to about 70%.

76. A pharmaceutical composition as in any one of claims 48-57, wherein the relative percent loading of the pharmaceutically active species in the porous material is between about 20% and about 90%.

77. A pharmaceutical composition as in any one of claims 48-57, wherein the relative percent loading of the pharmaceutically active species in the porous material is between about 70% and about 90%.
